(19) United States
(12) Patent Application Publication
(10) Pub. No.: US 2025/0257413 A1
(43) Pub. Date: Aug. 14, 2025

(54) METHODS AND COMPOSITIONS FOR MULTIPLEXED SINGLE-CELL 3D SPATIAL GENE EXPRESSION ANALYSIS IN PLANT TISSUE

(71) Applicant: SALK INSTITUTE FOR BIOLOGICAL STUDIES, LA JOLLA, CA (US)

(72) Inventors: Tatsuya Nobori, Norwich (GB); Joseph R. Ecker, Carlsbad, CA (US)

(73) Assignee: SALK INSTITUTE FOR BIOLOGICAL STUDIES, LA JOLLA, CA (US)

(21) Appl. No.: 18/998,032

(22) PCT Filed: Jul. 26, 2023
(86) PCT No.: PCT/US2023/071049

§ 371 Date: Jan. 23, 2025

Related U.S. Application Data
(60) Provisional application No. 63/392,392, filed on Jul. 26, 2022.

(51) Int. Cl. C12Q 1/6895; C12Q 1/682; C12Q 1/6841

(52) U.S. Cl. CPC C12Q 1/6895; C12Q 1/682; C12Q 1/6841; C12Q 2600/13; C12Q 2600/158; C12Q 2600/16

(57) ABSTRACT

The present disclosure provides a multiplexed fluorescence in situ hybridization method that enables single-cell and spatial analysis of gene expression in plant tissue in a transgene-free manner. The present disclosure provides methods and compositions of spatially mapping at least one gene in plant tissue in situ. Also provided is a kit for spatially mapping a plurality of genes in plant tissue in situ.

### CROSS-REFERENCE TO RELATED APPLICATIONS

This application claims the benefit of priority to U.S. Provisional Application No. 63/392,392 filed on Jul. 26, 2022, which is hereby incorporated by reference in its entirety.

### FIELD

The present disclosure generally relates to the field of spatial gene expression analysis in plants. More particularly, the present disclosure relates to methods and compositions for efficient spatial gene expression analysis in whole-mount tissue.

### BACKGROUND OF THE DISCLOSURE

Various types of cells comprising plants, as well as other organisms, process numerous developmental and environmental cues with cell-specific gene regulatory principles and thus manifest highly heterogeneous responses. Understanding how individual cells respond and interact with each other in the face of changing environments is the cornerstone of understanding tissue function. Single-cell transcriptomics technologies have been widely adopted in plant research, enabling the classification of cells into cell populations that share molecular features for the in-depth analysis of cell types and states (Cole et al., 2021; Seyfferth et al., 2021; Birnbaum, 2018). The increasing throughput and sensitivity in single-cell transcriptomics technologies offers the potential for tremendous granularity at which cells can be classified, but it will create new challenges in dealing with cell populations that the current histological and physiological understanding of plant cells cannot account for. While the single-cell transcriptomics have gained popularity in basic plant research and plant biotechnology, they are also unable to provide spatial relationships between cells.

Therefore, there is a need of understanding the identity and function of molecularly defined cell populations, and analyzing their spatial localization at the single-cell level in the plant tissue.

### SUMMARY OF THE DISCLOSURE

The present disclosure provides a method of spatially mapping gene expression of a plurality of genes in plant tissue in situ. In some embodiments, the method comprises fixing a plant tissue with a fixative. In some embodiments, the method comprises permeabilizing the plant tissue. In some embodiments, the method comprises hybridizing a plurality of DNA probes with target RNA molecules transcribed from at least one gene. In some embodiments, the method comprises amplifying said probes having barcodes by rolling circle amplification (RCA); detecting a plurality of amplified signals from said probes by a sequence-by-hybridization (SBH) chemistry, thereby identifying location of the target RNA molecules. In some embodiments, the method comprises obtaining three dimensional gene expression map with the plurality of the genes. In some embodiments, the method allows the interrogation of spatial regulation of complex cellular responses in the plant tissue during its developmental stages and/or during its exposure to stress.

The present disclosure provides a kit for spatially mapping gene expression of a plurality of genes in plant tissue in situ. In some embodiments, the kit comprises a fixative comprising a formalin, an acetic acid, and an alcohol. In some embodiments, the kit comprises a mixture of cell wall penetrating enzymes. In some embodiments, the kit comprises a T4 DNA ligase. In some embodiments, the kit comprises a plurality of DNA probes for a control gene, each of the DNA probes comprising a padlock probe and a primer, wherein the padlock probe and the primer have complementary sequences to form a circular structure. In some embodiments, the kit comprises a plurality of bridge probes, each of the bridge probes having a complementary sequence for the padlock probe. In some embodiments, the kit comprises a plurality of fluorescent probes, each of the fluorescent probes is hybridized with each of the bridge probes. In some embodiments, the kit comprises instructions on how to design the DNA probes, the bridge probes, and fluorescent probes, all of which are specific for a plurality of genes of interest.

In some embodiments, the target RNA molecules are expressed in the identified location. In some embodiments, the fixative comprises a formalin, an acetic acid, and an alcohol. In some embodiments, the alcohol is an ethanol. In some embodiments, a cell wall of the plant tissue is permeabilized with a cell wall degradation enzyme. In some embodiments, the cell wall degradation enzyme is a cellulase, a macerozme, pectinase, a glycosyl-dyrolase, an oxidoreductase, a lyase, an esterase, a polygalaturonase, a protease, or a xylanase. In some embodiments, the cell wall degradation enzyme is a cellulase, a macerozme, pectinase, or combinations thereof.

In some embodiments, the DNA probe comprises at least one gene-specific barcode. In some embodiments, the at least one barcode is specific for each of the target RNA molecules. In some embodiments, the target RNA molecules that are hybridized by the DNA probes are circularized by ligation. In some embodiments, the circularized target RNA molecules are amplified in situ by rolling circle amplification (RCA). In some embodiments, the amplified probes having the barcodes in DNA amplicons are detected. In some embodiments, at least one bridge probe is hybridized to at least one of the plurality of the amplified probes. In some embodiments, the at least one bridge probe is targeted by at least one fluorescent probe. In some embodiments, the at least one fluorescent probe is imaged by a plurality channel of a confocal microscope. In some embodiments, the bridge probes and the fluorescent probes are stripped away after imaging and re-hybridized to at least one of the plurality of DNA probes that are not previously hybridized. In some embodiments, the re-hybridized fluorescent probes are imaged by a plurality channel of a confocal microscope. In some embodiments, the imaging of the stripped and re-hybridized probes are repeated at least two times until all the barcodes are read. In some embodiments, each round of the imaging provides location information of each of the target RNA molecules hybridized with the DNA probes that are further hybridized with fluorescent probes.

In some embodiments, each round of the imaging detects expression of at least two genes. In some embodiments, at least four target RNA molecules are identified from the amplified signals from said probes per one round of an imaging. In some embodiments, at least two rounds of the imaging are performed in the plant tissue to identify location of the target RNA molecules. In some embodiments, the spatially mapped genes collected from at least two at least two rounds of imaging are mapped to locate the genes in the three dimensional gene expression map. In some embodiments, expression of at least 10 genes, at least 100 genes, at least 1,000 genes, or at least 10,000 genes are spatially mapped after the at least two rounds of imaging in plant tissue. In some embodiments, the plant tissue is whole-mount or sectioned.

In some embodiments, gene expression of a plurality of genes is spatially mapped in a plurality of plant cell types in the plant tissue.

### BRIEF DESCRIPTION OF THE DRAWINGS

FIGS. 1a-1f show whole-mount spatial mapping of root tip cell-type marker genes with plant hybridization-based targeted observation of gene expression map.

FIG. 1a shows that target mRNA molecules are hybridized by pairs of DNA probes (SNAIL probes) that harbor mRNA species-specific barcode sequences (pink bars) in fixed whole-mount tissue. Barcode-containing DNA probes are circularized by ligation (star) and amplified in situ by RCA. During amplification, amine-modified nucleotides are incorporated into the DNA amplicons (RCPs) and stably cross-linked with the cellular protein matrix using a non-reversible amine cross-linker. Amplified DNA barcodes are detected by sequence-by-hybridization (SBH) chemistry through multiple rounds of imaging. FIG. 1b shows sequence-by-hybridization (SBH) chemistry. Before each imaging round, four types of bridge probes are hybridized to a set of four DNA barcodes. Each bridge probe is then targeted by one of four fluorescent probes to be imaged. After imaging, bridge probes and fluorescent probes are stripped away, keeping RCPs in place. These steps are repeated until all the DNA barcodes are read. FIG. 1c shows representative images at different imaging rounds. The maximum exposure of 60 z planes of the same position in the tissue is displayed. Scale bar, 30 μm. FIG. 1d shows schematic representation of the root tip and UMAPs displaying root tip scRNA-seq data used in this study. In the UMAPs, cells are labelled with cell types (left) and regions (right). LRC, lateral root cap; QC, quiescent centre. FIGS. 1e and 1f show representative results from the imaging rounds 2 (FIGS. 1e) and 3 (FIG. 1f). Left, UMAPs showing expression patterns of target genes. The colors of the gene name labels correspond to the colors in the images below. Middle, 3D projections (upper) and optical sections (2D, lower) of whole-mount tissue images. Right, representative cross-section views of the middle part of the samples (transition/elongation zone). Scale bar, 25 μm.

FIGS. 2a-2g show single-cell and spatial analysis of 28 genes used in the study of the present disclosure.

FIG. 2a shows data analysis pipeline of the gene expression map for single-cell analysis. FIG. 2b shows 3D visualization of transcripts detected and decoded after image registration in a representative root tip (root 4). A middle section (z planes 90-120 of 208) of the image is displayed. Representative genes from each imaging round are shown. FIG. 2c shows violin plots showing the number of unique RNA molecules (left) and genes (right) detected in five root tip samples. FIG. 2d shows on the left panel, scatter plot comparing normalized bulk expression of each gene between two samples (root 1 and root 2) and correlation plot showing pair-wise correlation coefficients among five replicates on the left panel. FIG. 2e shows hierarchical clustering of cells of root 4 based on the relative expression of 28 genes. Cluster IDs are indicated at the bottom. RE, relative expression. FIG. 2f shows UMAP visualization of the clusters shown in FIG. 2e. FIG. 2g shows 3D visualization of transcripts colored by clusters in FIG. 2e and FIG. 2f in a representative root tip (root 4). A middle section (z planes 90-120 of 208) of the image is displayed. Scale bar, 25 μm (FIG. 2b, FIG. 2g).

FIG. 3 shows design of probes used in the gene expression map of the present disclosure. ID sequences are unique to different RNA species. Anchor sequence was included based on Gyllborg, D. et al. (Nucleic Acids Res. 48, e112 (2020)), but not used in the present study.

FIGS. 4a-4b show optimization of formamide concentration during SNAIL probe hybridization. FIG. 4a shows hybridization in 30% formamide showed higher target specificity. FIG. 4b shows images after stripping fluorescent probes. The four-color channels are shown in higher contrast than in FIG. 2b, and cell wall staining images are overlaid. Scale bars=25 μm. Three independent roots were tested with similar results.

FIGS. 5a-5c show validation of plant hybridization-based targeted observation of gene expression map. The mRNA of GFP was targeted with HCR (left) or the gene expression map (right) in ELTP:FLS2-GFP (FIG. 5a) and LBD16::FLS2-GFP (FIG. 5b) plants, which express GFP in endodermis and pericycle, respectively. Scale bars=25 μm. c, The gene expression map images that cover larger areas. (Left) ELTP: FLS2-GFP. (Right) LBD16::FLS2-GFP. Scale bar=100 μm. Three independent roots were tested with similar results.

FIGS. 6a-6c show whole-mount spatial mapping of root tip cell type marker genes predicted in scRNA-seq data with the gene expression map. FIGS. 6a and 6b show representative results from each imaging round. Left: UMAPs showing expression patterns of target genes. The colors of gene name labels correspond to the colors in the images below. Middle: 3D projections (top) and optical sections (2D, bottom) of whole-mount tissue images. Right: Representative cross-section views of the middle part of the samples (transition/elongation zone). FIG. 6c shows validated and predicted marker genes for QC and columella. 3D images were shown with cell wall staining. Scale bars=25 μm.

FIGS. 7a-7b show detailed analysis of the gene expression map images and magnified images of 2D optical section images in FIG. 6a and FIG. 6b, respectively. UMAP plots show expression patterns of target genes. The colors of gene name labels correspond to the colors in the images below. Scale bars=25 μm.

FIGS. 8a-8b show the magnified 2D optical section images of additional genes that are not shown in FIG. 1 and the gene expression map images for the additional genes. UMAP plots show expression patterns of target genes. The colors of gene name labels correspond to the colors in the images below. Scale bars=25 μm.

FIGS. 9a-9b show the magnified 2D optical section images of additional genes that are not shown in FIG. 1 and the gene expression map images for the additional genes. UMAP plots show expression patterns of target genes. The colors of gene name labels correspond to the colors in the images below. Scale bars=25 μm.

FIG. 10 shows the magnified 2D optical section images of additional genes that are not shown in FIG. 1 and the gene expression map images for the additional genes. UMAP plots show expression patterns of target genes. The colors of gene name labels correspond to the colors in the images below. Scale bars=25 μm.

FIG. 11 shows varying levels of expression of the genes targeted in this study. Bulk expression levels of genes (transcript per million) were calculated based on the root tip scRNA-seq data. The twenty-eight genes targeted in this study were presented in FIG. 11.

FIGS. 12a-12b show quantitative analysis of the gene expression map data across 14 rounds of imaging. FIG. 12a shows expression of genes labelled with Alexa Fluor 488/555/647 (FIG. 10). Data were shown as relative expression to R1. FIG. 12b shows expression of genes labelled with Alexa Fluor 750 (FIG. 10). Data were shown as relative expression to R2 as the data of R1 showed unusually weak signals. n=3 independent roots. Error bars indicate standard deviation.

FIGS. 13a-13c show spatial expression analysis of 28 genes with the gene expression map. FIG. 13a shows hierarchical clustering of 3608 cells from five root tips based on the relative expression (RE) of 28 genes. Leiden cluster IDs are indicated at the bottom. (FIGS. 13b-13c), UMAP visualization of the data shown in (FIG. 13a) colored by (FIG. 13b) Leiden clusters and (FIG. 13c) replicates.

FIGS. 14a-14b show the gene expression map across 14 rounds of imaging. FIG. 14a shows representative 2D optical sections from each imaging round. Magenta: AT3G46280 labeled with Alexa Fluor 555. Cyan: AT2G31310 labeled with Alexa Fluor 647. Green: AT5G57620 Alexa Fluor 488. FIG. 14b shows Red: AT3G10080 labeled with Alexa Fluor 750. Signal was not detected after the 9th round. Scale bars=25 μm.

FIG. 15 demonstrates diagram showing the key steps and timings of the gene expression map.

FIGS. 16a-16b show the gene expression map in Arabidopsis leaves. FIG. 16a shows 3D rendering of cell wall staining (leaf) and UBQ10 (right) images in a whole-mount leaf of Arabidopsis. FIG. 16b shows 2D the gene expression map image of cell wall staining (white) and UBQ10 (spotted). Scale bars=100 μm.

FIG. 17 lists 28 genes analyzed in the study described in Examples including FIG. 2e, and FIG. 11.

### DETAILED DESCRIPTION OF THE DISCLOSURE

### I. Definitions

Unless stated otherwise, all technical and scientific terms used herein have the same meaning as commonly understood by those of ordinary skill in the art to which the disclosure belongs. While the following terms are believed to be well understood by one of ordinary skill in the art, the following definitions are set forth to facilitate explanation of the presently disclosed subject matter. Although any methods and materials similar or equivalent to those described herein can be used in the practice or testing of the present disclosure, preferred methods and materials are described. The following terms are defined below. These definitions are for illustrative purposes and are not intended to limit the common meaning in the art of the defined terms.

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used in this specification, the term “and/or” is used in this disclosure to mean either “and” or “or” unless indicated otherwise.

Throughout this specification, unless the context requires otherwise, the words “comprise”, or variations such as “comprises” or “comprising”, will be understood to imply the inclusion of a stated element or integer or group of elements or integers but not the exclusion of any other element or integer or group of elements or integers.

As used in this application, the terms “about” and “approximately” are used as equivalents. Any numerals used in this application with or without about/approximately are meant to cover any normal fluctuations appreciated by one of ordinary skill in the relevant art.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids. In some embodiments, a fragment of a polypeptide or polynucleotide comprises at least 10%, 20%, 30%, 40%, 50%, 60%, 70%, 80%, 90%, 95%, 96%, 97%, 98%, or 99% of the entire length of the reference polypeptide or polynucleotide. In some embodiments, a polypeptide or polynucleotide fragment may contain 5, 10, 15, 20, 25, 30, 35, 40, 45, 50, 60, 70, 80, 90, 100, 200, 300, 400, 500, 600, 700, 800, 900, 1000, 2000 or more nucleotides or amino acids.

As used herein, the term “codon optimization” implies that the codon usage of a DNA or RNA is adapted to that of a cell or organism of interest to improve the transcription rate of said recombinant nucleic acid in the cell or organism of interest. The skilled person is well aware of the fact that a target nucleic acid can be modified at one position due to the codon degeneracy, whereas this modification will still lead to the same amino acid sequence at that position after translation, which is achieved by codon optimization to take into consideration the species-specific codon usage of a target cell or organism.

As used herein, the term “endogenous” or “endogenous gene,” refers to the naturally occurring gene, in the location in which it is naturally found within the host cell genome. “Endogenous gene” is synonymous with “native gene” as used herein. An endogenous gene as described herein can include alleles of naturally occurring genes that have been mutated, i.e. an endogenous gene could have been modified at some point by traditional plant breeding methods and/or next generation plant breeding methods.

As used herein, the term “exogenous” refers to a substance coming from some source other than its native source. For example, the terms “exogenous protein,” or “exogenous gene” refer to a protein or gene from a non-native source, and that has been artificially supplied to a biological system. As used herein, the term “exogenous” is used interchangeably with the term “heterologous,” and refers to a substance coming from some source other than its native source.

The terms “genetically engineered host cell,” “recombinant host cell,” and “recombinant strain” are used interchangeably herein and refer to host cells that have been genetically engineered. Thus, the terms include a host cell (e.g., bacteria, yeast cell, fungal cell, CHO, human cell, plant cell, protoplast derived from plant, callus, etc.) that has been genetically altered, modified, or engineered, such that it exhibits an altered, modified, or different genotype and/or phenotype (e.g., when the genetic modification affects coding nucleic acid sequences), as compared to the naturally-occurring host cell from which it was derived. It is understood that the terms refer not only to the particular recombinant host cell in question, but also to the progeny or potential progeny of such a host cell.

As used herein, the term “heterologous” refers to a substance coming from some source or location other than its native source or location. In some embodiments, the term “heterologous nucleic acid” refers to a nucleic acid sequence that is not naturally found in the particular organism. For example, the term “heterologous promoter” may refer to a promoter that has been taken from one source organism and utilized in another organism, in which the promoter is not naturally found. However, the term “heterologous promoter” may also refer to a promoter that is from within the same source organism, but has merely been moved to a novel location, in which said promoter is not normally located.

Heterologous gene sequences can be introduced into a target cell by using an “expression vector,” which can be a eukaryotic expression vector, for example a plant expression vector. Methods used to construct vectors are well known to a person skilled in the art and described in various publications. In particular, techniques for constructing suitable vectors, including a description of the functional components such as promoters, enhancers, termination and polyadenylation signals, selection markers, origins of replication, and splicing signals, are reviewed in the prior art. Vectors may include but are not limited to plasmid vectors, phagemids, cosmids, artificial/mini-chromosomes (e.g. ACE), or viral vectors such as baculovirus, retrovirus, adenovirus, adeno-associated virus, herpes simplex virus, retroviruses, bacteriophages. The eukaryotic expression vectors will typically contain also prokaryotic sequences that facilitate the propagation of the vector in bacteria such as an origin of replication and antibiotic resistance genes for selection in bacteria. A variety of eukaryotic expression vectors, containing a cloning site into which a polynucleotide can be operatively linked, are well known in the art and some are commercially available from companies such as Stratagene, La Jolla, Calif.; Invitrogen, Carlsbad, Calif.; Promega, Madison, Wis. or BD Biosciences Clontech, Palo Alto, Calif. In one embodiment the expression vector comprises at least one nucleic acid sequence which is a regulatory sequence necessary for transcription and translation of nucleotide sequences that encode for a peptide/polypeptide/protein of interest.

As used herein, the term “naturally occurring” as applied to a nucleic acid, a polypeptide, a cell, or an organism, refers to a nucleic acid, polypeptide, cell, or organism that is found in nature. The term “naturally occurring” may refer to a gene or sequence derived from a naturally occurring source. Thus, for the purposes of this disclosure, a “non-naturally occurring” sequence is a sequence that has been synthesized, mutated, engineered, edited, or otherwise modified to have a different sequence from known natural sequences. In some embodiments, the modification may be at the protein level (e.g., amino acid substitutions). In other embodiments, the modification may be at the DNA level (e.g., nucleotide substitutions).

As used herein, the term “nucleotide change” or “nucleotide modification” refers to, e.g., nucleotide substitution, deletion, and/or insertion, as is well understood in the art. For example, such nucleotide changes/modifications include mutations containing alterations that produce silent substitutions, additions, or deletions, but do not alter the properties or activities of the encoded protein or how the proteins are made. As another example, such nucleotide changes/modifications include mutations containing alterations that produce replacement substitutions, additions, or deletions, that alter the properties or activities of the encoded protein or how the proteins are made.

As used herein, the term “protein modification” refers to, e.g., amino acid substitution, amino acid modification, deletion, and/or insertion, as is well understood in the art.

As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the disclosure can be operably linked, either directly or indirectly, 5′ to the target mRNA, or 3′ to the target mRNA, or within the target mRNA, or a first complementary region is 5′ and its complement is 3′ to the target mRNA.

The terms “polynucleotide,” “nucleic acid,” and “nucleotide sequence,” used interchangeably herein, refers to a polymeric form of nucleotides of any length, either ribonucleotides or deoxyribonucleotides, or analogs thereof. This term refers to the primary structure of the molecule, and thus includes double-and single-stranded DNA, as well as double-and single-stranded RNA. This term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. It also includes modified nucleic acids such as methylated and/or capped nucleic acids, nucleic acids containing modified bases, backbone modifications, and the like. “Oligonucleotide” generally refers to polynucleotides of between about 5 and about 100 nucleotides of single-or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” “nucleic acid,” and “nucleotide sequence” should be understood to include, as applicable to the embodiments being described, single-stranded (such as sense or antisense) and double-stranded polynucleotides.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the phrases “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4: 2411-2418; De Almeida et al., (1989) Mol. Gen. Genetics 218: 78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11-17 (1988).

“Complementary” refers to the capacity for pairing, through base stacking and specific hydrogen bonding, between two sequences comprising naturally or non-naturally occurring bases or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369: 492-493 and Loakes et al., Nucleic Acids Res., 1994; 22: 4039-4043. Inosine (I) has also been considered in the art to be a universal base and is considered complementary to any A, C, U, or T. See Watkins and Santa Lucia, Nucl. Acids Research, 2005; 33 (19): 6258-6267.

As referred to herein, a “complementary nucleic acid sequence” is a nucleic acid sequence comprising a sequence of nucleotides that enables it to non-covalently bind to another nucleic acid in a sequence-specific, antiparallel, manner (i.e., a nucleic acid specifically binds to a complementary nucleic acid) under the appropriate in vitro and/or in vivo conditions of temperature and solution ionic strength.

Methods of sequence alignment for comparison and determination of percent sequence identity and percent complementarity are well known in the art. Optimal alignment of sequences for comparison can be conducted, e.g., by the homology alignment algorithm of Needleman and Wunsch, (1970) J. Mol. Biol. 48: 443, by the search for similarity method of Pearson and Lipman, (1988) Proc. Nat'l. Acad. Sci. USA 85: 2444, by computerized implementations of these algorithms (GAP, BESTFIT, FASTA, and TFASTA in the Wisconsin Genetics Software Package, Genetics Computer Group, 575 Science Dr., Madison, WI), by manual alignment and visual inspection (see, e.g., Brent et al., (2003) Current Protocols in Molecular Biology), by use of algorithms know in the art including the BLAST and BLAST 2.0 algorithms, which are described in Altschul et al., (1977) Nuc. Acids Res. 25:3389-3402; and Altschul et al., (1990) J. Mol. Biol. 215: 403-410, respectively. Software for performing BLAST analyses is publicly available through the National Center for Biotechnology Information. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, CA). Another alignment program is Sequencher (Gene Codes, Ann Arbor, Michigan), using default parameters, and MUSCLE (Multiple Sequence Comparison by Log-Expection; a computer software licensed as public domain).

Herein, the term “hybridize” refers to pairing between complementary nucleotide bases (e.g., adenine (A) forms a base pair with thymine (T) in a DNA molecule and with uracil (U) in an RNA molecule, and guanine (G) forms a base pair with cytosine (C) in both DNA and RNA molecules) to form a double-stranded nucleic acid molecule. (See, e.g., Wahl and Berger (1987) Methods Enzymol. 152: 399; Kimmel, (1987) Methods Enzymol. 152: 507). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a guide RNA molecule is considered complementary to a uracil (U), and vice versa. As such, when a G/U base-pair can be made at a given nucleotide position a protein-binding segment (dsRNA duplex) of a guide RNA molecule, the position is not considered to be non-complementary, but is instead considered to be complementary. It is understood in the art that the sequence of polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted.

The term “modified” refers to a substance or compound (e.g., a cell, a polynucleotide sequence, and/or a polypeptide sequence) that has been altered or changed as compared to the corresponding unmodified substance or compound.

“Isolated” refers to a material that is free to varying degrees from components which normally accompany it as found in its native state.

The term “gene edited plant, part or cell” as used herein refers to a plant, part or cell that comprises one or more endogenous genes that are edited by a gene editing system. The gene editing system generally comprises a targeting element and/or an editing element. The targeting element is capable of recognizing a target genomic sequence. The editing element is capable of modifying the target genomic sequence, e.g., by substitution or insertion of one or more nucleotides in the genomic sequence, deletion of one or more nucleotides in the genomic sequence, alteration of genomic sequences to include regulatory sequences, insertion of transgenes at a safe harbor genomic site or other specific location in the genome, or any combination thereof. The targeting element and the editing element can be on the same nucleic acid molecule or different nucleic acid molecules.

The term “plant part” includes differentiated and undifferentiated tissues including, but not limited to: plant organs, plant tissues, roots, stems, shoots, rootstocks, scions, stipules, petals, leaves, flowers, ovules, pollens, bracts, petioles, internodes, bark, pubescence, tillers, rhizomes, fronds, blades, stamens, fruits, seeds, tumor tissue and plant cells (e.g., single cells, protoplasts, embryos, and callus tissue). Plant cells include, without limitation, cells from seeds, suspension cultures, embryos, meristematic regions, callus tissue, leaves, roots, shoots, gametophytes, sporophytes, pollen and microspores. The plant tissue may be in a plant or in a plant organ, tissue or cell culture.

As used herein when discussing plants, the term “ovule” refers to the female gametophyte, whereas the term “pollen” means the male gametophyte.

As used herein, the term “plant tissue” refers to any part of a plant. Examples of plant organs include, but are not limited to the leaf, stem, root, root tip, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

As used herein, the term “phenotype” refers to the observable characters of an individual cell, cell culture, organism (e.g., a plant), or group of organisms which results from the interaction between that individual's genetic makeup (i.e., genotype) and the environment.

The terms “transgene” or “transgenic” as used herein refer to at least one nucleic acid sequence that is taken from the genome of one organism, or produced synthetically, and which is then introduced into a host cell or organism or tissue of interest and which is subsequently integrated into the host's genome by means of “stable” transformation or transfection approaches. In contrast, the term “transient” transformation or transfection or introduction refers to a way of introducing molecular tools including at least one nucleic acid (DNA, RNA, single-stranded or double-stranded or a mixture thereof) and/or at least one amino acid sequence, optionally comprising suitable chemical or biological agents, to achieve a transfer into at least one compartment of interest of a cell, including, but not restricted to, the cytoplasm, an organelle, including the nucleus, a mitochondrion, a vacuole, a chloroplast, or into a membrane, resulting in transcription and/or translation and/or association and/or activity of the at least one molecule introduced without achieving a stable integration or incorporation and thus inheritance of the respective at least one molecule introduced into the genome of a cell. The terms “transgene-free” refers to a condition that transgene is not present or found in the genome of a host cell or tissue or organism of interest.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, leaves, stems, roots, root tips, anthers, pistils, meristematic cells, axillary buds, ovaries, seed coat, endosperm, hypocotyls, cotyledons and the like. The term “plant organ” refers to plant tissue or a group of tissues that constitute a morphologically and functionally distinct part of a plant. “Progeny” comprises any subsequent generation of a plant.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

By “biologically active portion” is meant a portion of a full-length parent peptide or polypeptide which portion retains an activity of the parent molecule. As used herein, the term “biologically active portion” includes deletion mutants and peptides, for example of at least about 8, 9, 10, 11, 12, 13, 14, 15, 16, 17, 18, 19, 20, 21, 22, 23, 24, 25, 30, 40, 50, 60, 70, 80, 90, 100, 120, 150, 300, 400, 500, 600, 700, 800, 900 or 1000 contiguous amino acids, which comprise an activity of a parent molecule. Portions of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a peptide or polypeptide of the disclosure with proteinases such as endoLys-C, endoArg-C, endoGlu-C and staphylococcus V8-protease. The digested fragments can be purified by, for example, high performance liquid chromatographic (HPLC) techniques. Recombinant nucleic acid techniques can also be used to produce such portions.

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

The terms “growing” or “regeneration” as used herein mean growing a whole, differentiated plant from a plant cell, a group of plant cells, a plant part (including seeds), or a plant piece (e.g., from a protoplast, callus, or tissue part).

As used herein, the term “derived from” refers to the origin or source, and may include naturally occurring, recombinant, unpurified, or purified molecules. A nucleic acid or an amino acid derived from an origin or source may have all kinds of nucleotide changes or protein modification as defined elsewhere herein.

By “obtained from” is meant that a sample such as, for example, a nucleic acid extract or polypeptide extract is isolated from, or derived from, a particular source. For example, the extract may be isolated directly from plants.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein.

Proteins may be altered in various ways including amino acid substitutions, deletions, truncations, and insertions. Methods for such manipulations are generally known in the art.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another, Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine I, Lysine (K), Histidine (H); Acidic: Aspartic acid (D), Glutamic acid (E), Asparagine (N), Glutamine (Q). See also, Creighton, 1984. In addition, individual substitutions, deletions or additions which alter, add or delete a single amino acid or a small percentage of amino acids in an encoded sequence are also “conservatively modified variations.”

The term “plant” includes reference to whole plants, plant organs, plant tissues, and plant cells and progeny of same, but is not limited to angiosperms and gymnosperms such as Arabidopsis, potato, tomato, tobacco, alfalfa, lettuce, carrot, strawberry, sugarbeet, cassava, sweet potato, soybean, lima bean, pea, chick pea, maize (corn), turf grass, wheat, rice, barley, sorghum, oat, oak, eucalyptus, walnut, palm and duckweed as well as fern and moss. Thus, a plant may be a monocot, a dicot, a vascular plant reproduced from spores such as fern or a non-vascular plant such as moss, liverwort, hornwort and algae. The word “plant,” as used herein, also encompasses plant cells, seed, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds and microspores. Plants may be at various stages of maturity and may be grown in liquid or solid culture, or in soil or suitable media in pots, greenhouses or fields. Expression of an introduced leader, trailer or gene sequences in plants may be transient or permanent. A “selected plant species” may be, but is not limited to, a species of any one of these “plants.”

In the present disclosure, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, black raspberry, blueberry, broccoli, Brussel's sprouts, cabbage, cane berry, canola, cantaloupe, carrot, cassava, cauliflower, cedar, a cereal, celery, chestnut, cherry, Chinese cabbage, citrus, Clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, peach, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, wild strawberry, yams, yew, and zucchini.

Angiosperm is defined as vascular plants having seeds enclosed in an ovary. Angiosperms are seed plants that produce flowers that bear fruits. Angiosperms are divided into dicotyledonous and monocotyledonous plants.

Dicotyledonous plant (Dicot) is defined as a flowering plant whose embryos have two seed halves or cotyledons, branching leaf veins, and flower parts in multiples of four or five. Examples of dicots include but are not limited to, Eucalyptus, Populus, Liquidamber, Acacia, teak, mahogany, tobacco, Arabidopsis, tomato, potato sugar beet, broccoli, cassava, sweet potato, pepper, poinsettia, bean, rapeseed/canola, alfalfa, radish, crimson clover, field pennycress, soybean, carrot, strawberry, lettuce, oak, maple, walnut, rose, mint, squash, daisy, geranium, avocado, cotton/cottonseed and cactus.

Thlaspi arvense, known by the common name field pennycress (aka pennycress), is a flowering plant in the cabbage family Brassicaceae. CoverCress is a new oilseed crop grown over winter between normal full season corn and soybeans. CoverCress was developed from pennycress. Low fiber pennycress lines are provided in U.S. Pat. No. 10,709,151, which is assigned to CoverCress Inc.

Monocotyledonous Plant (Monocot) is defined as a flowering plant having embryos with one cotyledon or seed leaf, parallel leaf veins, and flower parts in multiples of three. Examples of monocots include, but are not limited to turfgrass, corn/maize, rice, oat, annual ryegrass, wheat, barley, sorghum, orchid, iris, lily, onion, and palm. Examples of turfgrass include, but are not limited to Agrostis spp. (bentgrass species including colonial bentgrass and creeping bentgrasses), Poa pratensis (Kentucky bluegrass), Lolium spp. (ryegrass species including annual ryegrass and perennial ryegrass), Festuca arundinacea (tall fescue) Festuca rubra commutata (Chewings fescue), Cynodon dactylon (bermudagrass, Pennisetum clandestinum (kikuyu grass), Stenotaphrum secundatum (St. Augustine grass), Zoysia japonica (zoysia grass), and Dichondra micrantha.

A wide variety of plants and plant cell systems may be targeted for the multiplexed spatial gene expression analysis of the present disclosure. In embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, grape, peach, pear, plum, raspberry, black raspberry, blackberry, cane berry, cherry, avocado, strawberry, wild strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). In some embodiments, fruit crops such as tomato, apple, peach, pear, plum, raspberry, black raspberry, blackberry, cane berry, cherry, avocado, strawberry, wild strawberry, grape and orange.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “genotype” refers to the genetic makeup of an individual cell, cell culture, tissue, organism (e.g., a plant), or group of organisms.

As used herein, the term “allele(s)” means any of one or more alternative forms of a gene, all of which alleles relate to at least one trait or characteristic. In a diploid cell, the two alleles of a given gene occupy corresponding loci on a pair of homologous chromosomes. Quantitative Trait Loci (QTLs) are genomic regions that may comprise one or more genes or regulatory sequences. It is in some instances more accurate to refer to “haplotype” (i.e. an allele of a chromosomal segment) instead of “allele”, however, in those instances, the term “allele” should be understood to comprise the term “haplotype”. Alleles are considered identical when they express a similar phenotype. Differences in sequence are possible but not important as long as they do not influence phenotype.

As used herein, the term “locus” (plural: “loci”) refers to any site that has been defined genetically. A locus may be a gene, or part of a gene, or a DNA sequence that has some regulatory role, and may be occupied by different sequences.

As used herein, the term “molecular marker” or “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Mapping of molecular markers in the vicinity of an allele is a procedure which can be performed quite easily by the average person skilled in molecular-biological techniques which techniques are for instance described in Lefebvre and Chevre, 1995; Lorez and Wenzel, 2007, Srivastava and Narula, 2004, Meksem and Kahl, 2005, Phillips and Vasil, 2001. General information concerning AFLP technology can be found in Vos et al. (1995, AFLP: a new technique for DNA fingerprinting, Nucleic Acids Res. 1995 Nov. 11; 23 (21): 4407-4414).

As used herein, the term “hemizygous” refers to a cell, tissue or organism in which a gene is present only once in a genotype, as a gene in a haploid cell or organism, a sex-linked gene in the heterogametic sex, or a gene in a segment of chromosome in a diploid cell or organism where its partner segment has been deleted.

As used herein, the term “heterozygote” refers to a diploid or polyploid individual cell or plant having different alleles (forms of a given gene) present at least at one locus.

As used herein, the term “heterozygous” refers to the presence of different alleles (forms of a given gene) at a particular gene locus.

As used herein, the term “homozygote” refers to an individual cell or plant having the same alleles at one or more loci.

As used herein, the term “homozygous” refers to the presence of identical alleles at one or more loci in homologous chromosomal segments.

As used herein, the term “homologous” or “homolog” is known in the art and refers to related sequences that share a common ancestor or family member and are determined based on the degree of sequence identity. The terms “homology”, “homologous”, “substantially similar” and “corresponding substantially” are used interchangeably herein. Homologs usually control, mediate, or influence the same or similar biochemical pathways, yet particular homologs may give rise to differing phenotypes. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared.

The term “homolog” is sometimes used to apply to the relationship between genes separated by the event of speciation (see “ortholog”) or to the relationship between genes separated by the event of genetic duplication (see “paralog”).

The term “homeolog” refers to a homeologous gene or chromosome, resulting from polyploidy or chromosomal duplication events. This contrasts with the more common ‘homolog’, which is defined immediately above.

The term “ortholog” refers to genes in different species that evolved from a common ancestral gene by speciation. Normally, orthologs retain the same function in the course of evolution. Identification of orthologs is critical for reliable prediction of gene function in newly sequenced genomes.

The term “paralog” refers to genes related by duplication within a genome. While orthologs generally retain the same function in the course of evolution, paralogs can evolve new functions, even if these are related to the original one.

“Homologous sequences” or “homologs” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. The degree of sequence identity may vary, but in one embodiment, is at least 50% (when using standard sequence alignment programs known in the art), at least 60%, at least 65%, at least 70%, at least 75%, at least 80%, at least 85%, at least 90%, at least about 91%, at least about 92%, at least about 93%, at least about 94%, at least about 95%, at least about 96%, at least about 97%, at least about 98%, or at least 98.5%, or at least about 99%, or at least 99.5%, or at least 99.8%, or at least 99.9%. Homology can be determined using software programs readily available in the art, such as those discussed in Current Protocols in Molecular Biology (F. M. Ausubel et al., eds., 1987) Supplement 30, section 7.718, Table 7.71. Some alignment programs are Mac Vector (Oxford Molecular Ltd, Oxford, U.K.) and ALIGN Plus (Scientific and Educational Software, Pennsylvania). Other non-limiting alignment programs include Sequencher (Gene Codes, Ann Arbor, Michigan), AlignX, and Vector NTI (Invitrogen, Carlsbad, CA).

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification.

Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T and G/C content) of primer. A pair of bi-directional primers consists of one forward and one reverse primer as commonly used in the art of DNA amplification such as in PCR amplification.

A probe comprises an identifiable, isolated nucleic acid that recognizes a target nucleic acid sequence. A probe includes a nucleic acid that is attached to an addressable location, a detectable label or other reporter molecule and that hybridizes to a target sequence. Typical labels include radioactive isotopes, enzyme substrates, co-factors, ligands, chemiluminescent or fluorescent agents, haptens, and enzymes. Methods for labelling and guidance in the choice of labels appropriate for various purposes are discussed, for example, in Sambrook et al. (ed.), Molecular Cloning: A Laboratory Manual, 2^{nd} ed., vol. 1-3, Cold Spring Harbor Laboratory Press, Cold Spring Harbor, NY, 1989 and Ausubel et al. Short Protocols in Molecular Biology, 4^{th} ed., John Wiley & Sons, Inc., 1999.

Methods for preparing and using nucleic acid probes and primers are described, for example, in Sambrook et al. (ed.), Molecular Cloning: A Laboratory Manual, 2^{nd} ed., vol. 1-3, Cold Spring Harbor Laboratory Press, Cold Spring Harbor, NY, 1989; Ausubel et al. Short Protocols in Molecular Biology, 4^{th} ed., John Wiley & Sons, Inc., 1999; and Innis et al. PCR Protocols, A Guide to Methods and Applications, Academic Press, Inc., San Diego, CA, 1990. Amplification primer pairs can be derived from a known sequence, for example, by using computer programs intended for that purpose such as PRIMER (Version 0.5, 1991, Whitehead Institute for Biomedical Research, Cambridge, MA). One of ordinary skill in the art will appreciate that the specificity of a particular probe or primer increases with its length. Thus, in order to obtain greater specificity, probes and primers can be selected that comprise at least 20, 25, 30, 35, 40, 45, 50 or more consecutive nucleotides of a target nucleotide sequences.

Methods for designing PCR primers and PCR cloning are generally known in the art and are disclosed in Sambrook et al. (2001) Molecular Cloning: A Laboratory Manual (3rd ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

As used herein, the term “barcode” is a label, or identifier, that conveys or is capable of conveying information (e.g., information about an analyte in a sample). Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier. Barcodes can spatially-resolve molecular components found in biological samples, for example, at single-cell resolution. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

As used herein, the term “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60%, at least 70%, at least 80%, or at least 90% of their individual bases are complementary to one another.

As used herein, the term “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

As used herein, the term “primer extension” refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence (e.g., capture domain) can be used as a template for single-strand synthesis of a corresponding cDNA molecule.

The terms “detectable label,” “optical label,” and “label” are used interchangeably herein to refer to a directly or indirectly detectable moiety that is associated with (e.g., conjugated to) a molecule to be detected, e.g., a probe for in situ assay or an analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties.

In some embodiments, a plurality of detectable labels can be attached to a detectably labeled probe. For example, detectable labels can be incorporated during nucleic acid polymerization or amplification (e.g., Cy5®-labeled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™ Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue@, Cascade Yellow™, CCF2 (GeneBLAzer™), CFP (Cyan Fluorescent Protein), CFP/YFP FRET, Chromomycin A3, CI-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF@-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM@ 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue@, 4-Methylumbelliferone, Mithramycin, MitoTracker® Green, MitoTracker® Orange, MitoTracker® Red, NBD (amine), Nile Red, Oregon Green® 488,Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As used herein, the term “whole-mount plant tissue” or “whole mount plant tissue” refers to the intact condition of the plant tissue without sectioning or slicing into thin layer(s). In some embodiments, whole-mount refers to an entire organism or structure that is small enough or thin enough to not require sectioning to reveal the structure.

### Overview

In the field of plant research, the primary method for spatially mapping cell population marker genes identified through single-cell transcriptome analysis has been the use of transgenic reporter lines. These lines express fluorescent proteins under the predicted promoter region of specific genes, visualizing the expression of one gene per transgenic line. However, this approach is not without limitations, particularly when studying cells within complex tissues:

At first, cell types or states cannot always be accurately defined solely by the expression of a single gene; rather, they are often determined by the intricate interactions and interplays of multiple genes.

Second, spatially mapping only a single gene or a few genes presents challenges in simultaneously analyzing multiple cell types or states. Understanding the interactions between these diverse cell types or states is crucial for gaining deeper insights into their functionalities.

Third, the process of generating transgenic plants is time-consuming, hampering the efficiency of research efforts.

Fourth, heterologous expression of fluorescent proteins may not necessarily represent the actual expression of the gene due to the lack of native genomic context, including critical enhancer-promoter interactions.

An alternative approach, in situ hybridization, is also popular for spatial gene expression analysis in plants. While it can address some of the above limitations, it suffers from limited multiplexing capacity, restricting the simultaneous analysis of a large number of genes.

Thus, there is a need to conduct spatial gene expression analysis with a large number of genes at single-cell resolution for a more comprehensive understanding of the function of different cell types or states, as well as their interactions with other cells and the environment. By overcoming the constraints of visualizing one gene at a time, this advanced technique holds the potential to unlock a deeper understanding of the complexities of plant biology.

Spatial transcriptomics technologies offer promising solutions to address the challenges faced in understanding the molecular details and spatial location of cells within complex tissues. Methods using spatially barcoded arrays or imaging-based, highly multiplexed single-molecule fluorescence in situ hybridization, have been developed to study the expression patterns of numerous genes, ranging from dozens to the entire transcriptome with spatial information, from tissue regions down to single-cell levels. These methods have been successfully integrated into plant research, opening new avenues for investigation.

Combining spatial transcriptomics with single-cell transcriptomics can delve into the spatial organization of different cell types and states within plants, offering detailed insights into their functions. However, this approach poses specific challenges in plants and other organisms. Notably, tissue types amenable to spatial transcriptomics experiments are limited to thin sections, often single-cell layers. This limitation proves troublesome, especially when studying crucial organs like the root tip, which plays a vital role in plant growth, nutrient acquisition, and interactions with microbes. Sectioning such small tissues leads to information loss from other parts of the tissue that may contain relevant cell types or states of interest. Moreover, important environmental information, such as microbial colonization, can also be lost by the sectioning process.

While one potential solution involves sampling serial sections and conducting multiple experiments, followed by the complex 3D reconstruction of 2D data, this approach proves costly and seldom affordable due to the expenses associated with spatial transcriptomics technologies.

To address these limitations, the present disclosure presents plant hybridization-based targeted observation of gene expression map, a cost-effective single-cell spatial gene expression analysis method capable of simultaneously mapping dozens of genes in plant tissue. By offering a low-cost alternative, the gene expression map aims to empower researchers to overcome the challenges of spatial transcriptomics in plants, enabling a more comprehensive understanding of plant biology and paving the way for exciting discoveries in the field. The plant hybridization-based targeted observation of gene expression map is a multiplexed fluorescence in situ hybridization method that enables single-cell and spatial analysis of gene expression in 3D plant tissue.

### Plant Hybridization-Based Targeted Observation of Gene Expression Map

The present disclosure teaches that the gene expression map can offer an opportunity to examine dozens of plant genes simultaneously, revealing vital information about which cells express those genes, how cells influence each other, and how tissue architecture influences those cells. This innovative technology empowers researchers to gain valuable insights into how genes are expressed, how cells influence one another, and how tissue structure plays a crucial role in shaping cellular behaviors.

Unlike existing imaging techniques, which are limited to observing only a small number of genes in one type of plant tissue and require altering the plant's genetic makeup by creating transgenic lines, the gene expression map circumvents these challenges. With its plant hybridization-based targeted observation of gene expression map approach, the present disclosure provides a novel tool to study dozens of genes in a single experiment, without the need for time-consuming genetic manipulations of the plant.

By unlocking a deeper understanding of gene expression patterns and cellular interactions, this gene expression map technology may help enhance crop improvement strategies, predict how plants might respond to climate change, and pave the way for numerous other discoveries in the realm of plant biology.

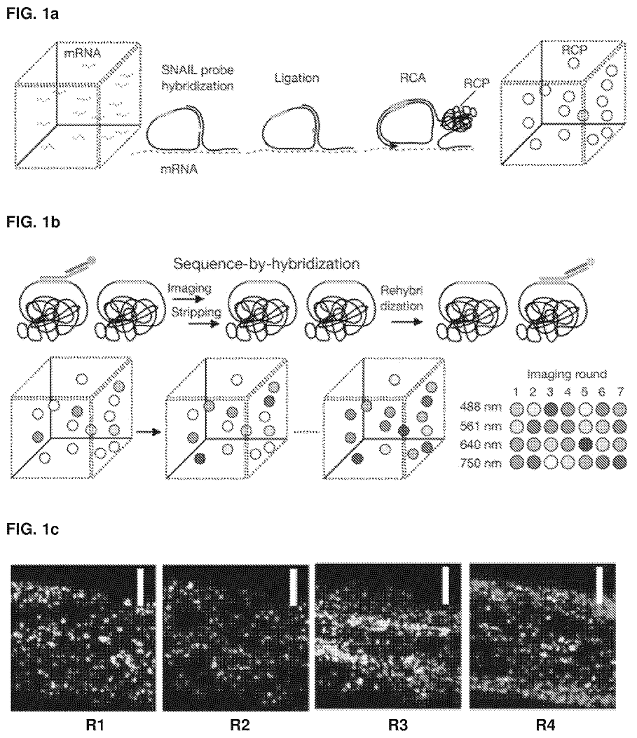

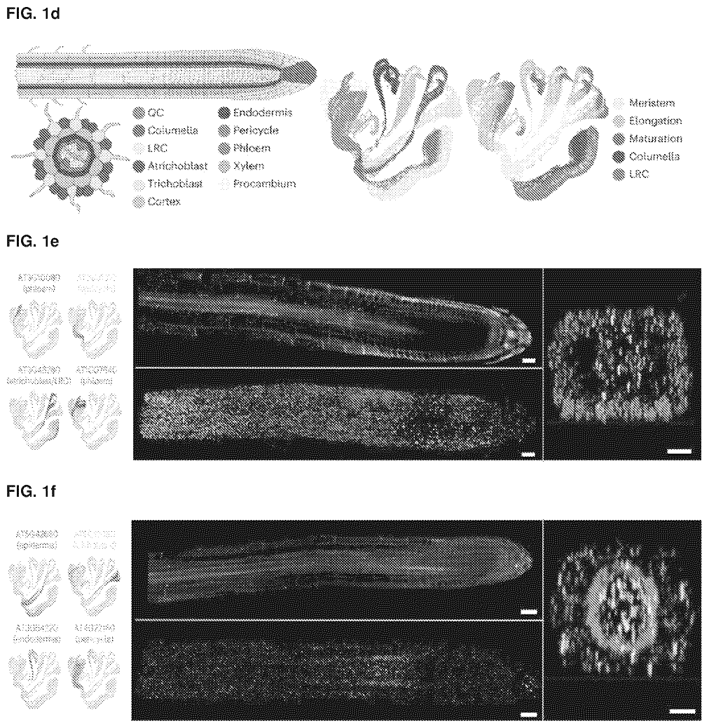

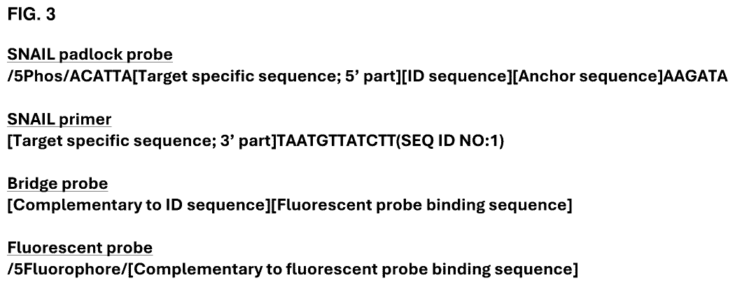

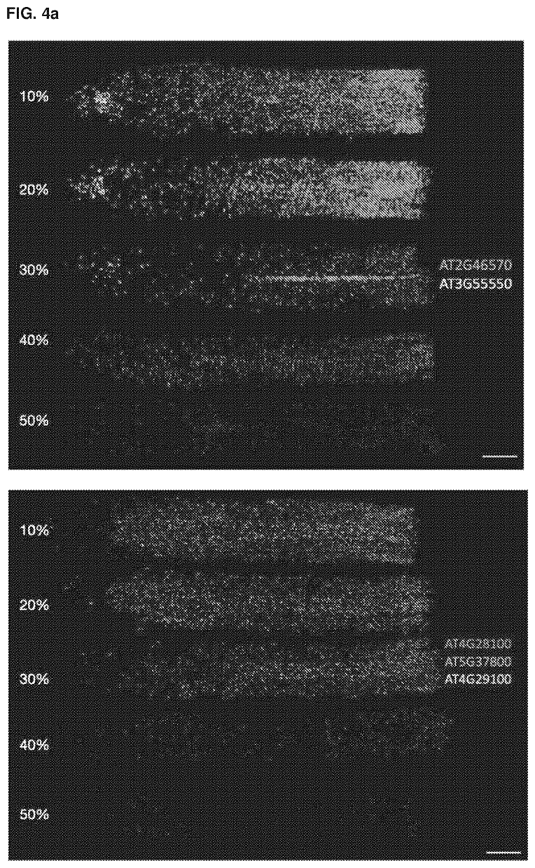

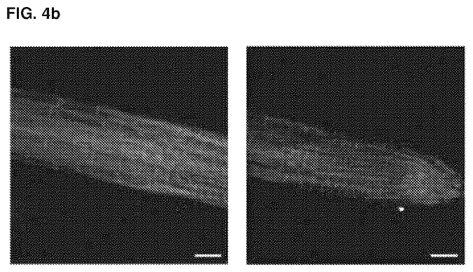

The present disclosure teaches that the gene expression map builds upon the in situ hybridization techniques in plants, combined with advancements in in situ sequencing technologies primarily developed in the field of neuroscience. The process involves fixing plant tissues and employing DNA probes, such as intramolecular ligation probes or SNAIL (Splint Nucleotide Assisted Intramolecular Ligation) probes, which carry gene-specific barcodes. These probes are hybridized to target messenger RNA molecules within the tissue (FIG. 1a and FIG. 3). Notably, the hybridization conditions have been optimized to ensure high target specificity (FIG. 4a).

Subsequently, the DNA barcodes are amplified in situ, creating a high signal-to-noise ratio that allows efficient signal detection even in cleared tissues. To identify the location of mRNA molecules, a sequence-by-hybridization (SBH) chemistry is employed, which targets the barcode sequences of DNA amplicons across sequential rounds of probing, imaging, and stripping (FIG. 1b).

In each imaging round, four targets are detected using each of the four channels of a confocal microscope. Following imaging, fluorescent detection probes are stripped off (FIG. 4b), paving the way for the next round of hybridization to target a fresh set of four genes (FIGS. 1b and 1c). The utilizing of SBH chemistry can detect amplified DNA probes in situ demonstrated that the signal remains detectable for at least ten cycles.

The present disclosure teaches features of the gene expression map, which make this technology a unique and valuable tool for a broad plant science community.

First, the gene expression map can spatially map multiple genes in t plant tissue. The plant tissues can be whole-mount or sectioned, and the gene expression map can provide multiplexed gene expression with spatial information.

Second, the gene expression map can be applied to any plant species, including ones that cannot be transformed. Also, the gene expression map is highly compatible with genetic approaches as it can directly tap into the ample mutant resources of model plants. This technology is transgene-free.

Third, researchers can easily follow straightforward protocols with a standard molecular biology setup and a confocal microscope, which will allow for the gene expression map experiment. Sample preparation takes about five days, comparable to standard in situ hybridization experiments.

Fourth, this technology is economical with low cost for 96-gene expression map experiment, for example, an order of magnitude lower than commercial spatial transcriptomics assays with similar gene-plex (such as Molecular Cartography). On top of the lower cost, the gene expression map can analyze in 3D whole-mount tissues, which is impossible with any commercially available spatial transcriptomics platforms that can use only tissue slices.

The gene expression map can dramatically accelerate the analysis of cell populations identified in single-cell transcriptomics by allowing researchers to spatially map dozens of candidate marker genes in their plant species of interest without generating transgenic lines. Beyond cell typing, the gene expression map can offer unique opportunities to interrogate spatial regulation of complex cellular responses in plant tissue during stress and development.

The present disclosure teaches 3D gene expression visualization with multi-gene profiling via the gene expression map.

### Method for Multiplexed Single-Cell 3D Spatial Gene Expression Analysis in Plant Tissue Using the Gene Expression Map of the Present Disclosure

The present disclosure provides a multiplexed fluorescence in situ hybridization method that enables single-cell and spatial analysis of gene expression in plant tissue in a transgene-free manner using the gene expression map.

The present disclosure teaches use of the gene expression map to simultaneously analyse dozes of cell-type marker genes in Arabidopsis roots. The present disclosure teaches successful identification of major cell types, demonstrating that this method can substantially accelerate the spatial mapping of marker genes defined in single-cell RNA-sequencing datasets in complex plant tissue.

The present disclosure provides methods of spatially mapping gene expression of a plurality of genes in plant tissue in situ. In some embodiments, the method comprises (i) fixing a plant tissue with a fixative; (ii) permeabilizing the plant tissue; (iii) hybridizing a plurality of DNA probes with target RNA molecules transcribed from at least one gene; (iv) amplifying said probes having barcodes by rolling circle amplification (RCA); (v) detecting a plurality of amplified signals from said probes by a sequence-by-hybridization (SBH) chemistry, thereby identifying location of the target RNA molecules; and (vi) obtaining three dimensional gene expression map with the plurality of the genes.

### (i) Fixation

The terms “fixing” or “fixation” refer to the preservation process of biological material, such as tissues, cells, organelles, molecules, etc., to prevent decay and degradation. Fixation is achieved through various protocols available for this purpose. The process involves treating the sample with a fixation reagent, which contains at least one fixative. The duration of sample contact with the fixation reagent can vary widely and is influenced by factors such as temperature, the nature of the sample, and the specific fixative(s) used. For example, a tissue sample can be contacted by a fixation reagent for 24 or less hours, 18 or less hours, 12 or less hours, 8 or less hours, 6 or less hours, 4 or less hours, 2 or less hours, 1 or less hour, 45 or less minutes, 30 or less minutes, 25 or less minutes, 20 or less minutes, 15 or less minutes, 10 or less minutes, 5 or less minutes, or 2 or less minutes. In some embodiments, a sample can be contacted by a fixation reagent for a period of time in a range of from 5 minutes to 24 hours.

In some embodiments, a cellular or tissue sample can be contacted by a fixation reagent at various temperatures, depending on the protocol and the reagent used. For example, in some embodiments, a cellular or tissue sample can be contacted by a fixation reagent at a temperature ranging from −25° C. to 55° C. In some embodiments, a sample can be contacted by a fixation reagent at a temperature of −25 to −20° C., −20 to −15° C., −15 to −10° C., −10 to −5° C., −5 to 0°° C., 0 to 5° C., 5 to 10° C., 10 to 15° C., 15 to 20° C., 20 to 25° C., 25 to 30° C., 30 to 35° C., 35 to 40° C., 40 to 45°° C., 45 to 50° C., or 50 to 55° C. In some embodiments, a sample can be contacted by a fixation reagent at-20° C., 4° C., room temperature (22-25° C.), 30° C., 37° C., or 42° C.

Any convenient fixation reagent can be used. Common fixation reagents include crosslinking fixatives, precipitating fixatives, oxidizing fixatives, mercurials, and the like. Crosslinking fixatives chemically join two or more molecules by a covalent bond and a wide range of cross-linking reagents can be used. Examples of suitable cross-liking fixatives include but are not limited to aldehydes (e.g., formaldehyde, also commonly referred to as “paraformaldehyde” and “formalin”; glutaraldehyde; etc.), imidoesters, NHS (N-Hydroxysuccinimide) esters, and the like. Examples of suitable precipitating fixatives include but are not limited to alcohols (e.g., methanol, ethanol, etc.), acetone, acetic acid, etc. In some embodiments, the fixative is formaldehyde (i.e., paraformaldehyde or formalin). A suitable final concentration of formaldehyde in a fixation reagent is 0.1 to 30%.

A fixation reagent can contain more than one fixative in any combination. For example, in some embodiments the cellular or tissue sample is contacted with a fixation reagent containing both formaldehyde and glutaraldehyde. In some embodiments, the fixative comprises a formalin, an acetic acid, and an alcohol. In further embodiments, the alcohol is an ethanol.

In some embodiments, the tissue sample are immersed in FAA (about 16% v/v formaldehyde, about 5% v/v acetic acid and about 50% ethanol) for 1 h at room temperature.

(ii) Permeabilization

The terms “permeabilization” or “permeabilize” as used herein refer to the process of rendering the cells (cell membranes etc.) of a sample permeable to experimental reagents such as nucleic acid probes, antibodies, chemical substrates, etc. Any convenient method and/or reagent for permeabilization can be used. Suitable permeabilization reagents include detergents (e.g., Saponin, Triton X-100, Tween-20, etc.), organic fixatives (e.g., acetone, methanol, ethanol, etc.), enzymes, etc.

In some embodiments, a sample can be contacted by a permeabilization reagent for a wide range of times, which can depend on the temperature, the nature of the sample, and on the permeabilization reagent(s). For example, a tissue sample can be contacted by a permeabilization reagent for 24 or more hours, 24 or less hours, 18 or less hours, 12 or less hours, 8 or less hours, 6 or less hours, 4 or less hours, 2 or less hours, 1 or less hour, 45 or less minutes, 30 or less minutes, 25 or less minutes, 20 or less minutes, 15 or less minutes, 10 or less minutes, 5 or less minutes, or 2 or less minutes. A sample can be contacted by a permeabilization reagent at various temperatures, depending on the protocol and the reagent used. For example, in some embodiments, a cellular or tissue sample can be contacted by a permeabilization reagent at a temperature ranging from −85° C. to 55° C. In some embodiments, a sample can be contacted by a permeabilization reagent at a temperature of −85 to −80° C., −80 to −75° C., −75 to −70° C., −70 to −65° C., −65 to −60°° C., −60 to −55° C., −55 to 40° C., −40 to-35° C., −35 to −30°° C., −30 tov25° C., −25 to −20° C., −20 to −15° C., −25 to −20°° C., −20 to −15° C., −25 to −20° C., −20 to −15° C., −25 to −20° C., −20 to −15° C., −15 to −10°° C.,-10 to −5° C., −5 to 0° C., 0 to 5° C., 5 to 10° C., 10 to 15° C., 15 to 20° C., 20 to 25° C., 25 to 30° C., 30 to 35° C., 35 to 40°° C., 40 to 45°° C., 45 to 50° C., or 50 to 55° C. In some embodiments, a sample can be contacted by a fixation reagent at-80° C.,-20° C., 4° C., room temperature (22-25° C.), 30° C., 37°° C., or 42°° C.

In some embodiments, a sample is contacted with an enzymatic permeabilization reagent. Enzymatic permeabilization reagents that permeabilize a cellular or tissue sample by partially or entirely degrading extracellular matrix or surface proteins that hinder the permeation of the cellular or tissue sample by assay reagents. Thus, cell wall is degraded by treatment of an enzymatic permeabilization reagent or a plurality of enzymatic permeabilization reagents. In some embodiments, a cell wall of the plant tissue is permeabilized with enzymatic permeabilization reagents, which includes cell wall degradation enzymes.

In some embodiments, the enzymatic permeabilization reagent is a cell wall degradation enzymes, which is a cellulase, a macerozme, pectinase, a glycosyl-dyrolase, an oxidoreductase, a lyase, an esterase, a polygalaturonase, a protease, or a xylanase.

In some embodiments, the cell wall degradation enzyme is a cellulase, a macerozme, pectinase, or combinations thereof.

Contact with an enzymatic permeabilization reagent can take place at any point after fixation and prior to target detection. The cell wall degradation enzyme can be performed over a range of times at a range of temperatures, over a range of enzyme concentrations that are empirically determined for each cell type or tissue type under investigation. For examples, a cellular or tissue sample can be contacted by proteinase K for 1 or less hour, 30 or less minutes, 25 or less minutes, 20 or less minutes, 15 or less minutes, 10 or less minutes, 5 or less minutes, or 2 or less minutes. A cellular or tissue sample can be contacted by 1 ug/ml or less, 2 ug/m or less 1, 4 ug/ml or less, 8 ug/ml or less, 10 ug/ml or less, 20 ug/ml or less, 30 ug/ml or less, 50 ug/ml or less, or 100 ug/ml or less enzyme. A cellular or tissue sample can be contacted by a cell wall degradation enzyme at a temperature ranging from 4° C. to 55° C. In some embodiments, a cellular or tissue sample can be contacted by cell wall degradation enzyme at a temperature of 4° C., room temperature (22-25° C.), 37° C., or 42° C.

Contact of a cellular or tissue sample with at least a fixation reagent and a permeabilization reagent results in the production of a fixed and permeabilized cellular or tissue sample.

The present disclosure teaches that plant tissues are fixed with the FAA fixative and dehydrated with a series of ethanol washes. It is critical to permeabilize the plant cell wall, so that the DNA probes and enzymes used in the following steps can get into the entire tissue; but digesting too much can cause tissue collapse. For tissue permeabilization, a cocktail of cell wall degradation enzymes (CWDEs; cellulase, macerozyme, and pectinase) was used in an optimized concentration and treatment strategy. Tissues are incubated in CWDEs for 5 min on ice then at room temperature for 30 min.

(iii) Hybridization

In some embodiments, the method taught herein includes a hybridization step, which comprises the pairing of substantially complementary or complementary nucleic acid sequences within two different molecules; one of which is the endogenous analyte (such as DNA, RNA or protein) or the labelling agent (e.g., reporter oligonucleotide attached thereto) and the other of which can be another endogenous molecule or an exogenous molecule such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60%, at least 70%, at least 80%, at least 90%, at least 95% or at least 99% of their individual bases are complementary to one another.

Various probes and probe sets can be hybridized to an endogenous analyte and/or a labelling agent and each probe may comprise one or more barcode sequences. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PUSH (Proximity Ligation in situ Hybridization) probe set, and RNA-templated ligation probes. The specific probe or probe set design can vary. In some embodiments, the barcoded probe is the padlock probe. In some embodiments, the barcoded probe is the gapped padlock probe. In some embodiments, the barcoded probe is the SNAIL probe.

The present disclosure also teaches that DNA probes with gene-specific barcodes (based on the SNAIL probe design from STARmap with modification) are specifically hybridized on target mRNA molecules, circularized by DNA ligation, and amplified by a DNA polymerase in situ. The hybridization condition has been optimized to allow high target specificity. The amplification of DNA barcodes provides high signal/noise ratio, enabling signal detection from cleared tissue. Tissues are cleared with the plant-optimized clearing solution ClearSee (Kurihara et al., 2015, Development).

### (iv) Ligation

In some embodiments, the method taught herein includes a ligation step. A ligation product is formed between two or more nucleic acid such as genomic DNA or mRNA. In some embodiments, the ligation product is formed between an endogenous analyte and a labelling agent. In some embodiments, the ligation product is formed between two or more labelling agent. In some embodiments, the ligation product is an intramolecular ligation of an endogenous analyte. In some embodiments, the ligation product is an intramolecular ligation of a labelling agent or probe, for example, the circularization of a circularizable probe or probe set upon hybridization to a target sequence. The target sequence can be comprised in an endogenous analyte (e.g., nucleic acid such as genomic DNA or mRNA) or a product thereof (e.g., cDNA from a cellular mRNA transcript), or in a labelling agent (e.g., the reporter oligonucleotide) or a product thereof.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244which is hereby incorporated by reference in its entirety. In some embodiments, the probe set is a SNAIL probe set. See, e.g., U.S. Pat. Pub. 20190055594, which is hereby incorporated by reference in its entirety.

In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, a probe such as a padlock probe may be used to analyze a reporter oligonucleotide, which may be generated using proximity ligation or be subjected to proximity ligation. In some examples, the reporter oligonucleotide of a labelling agent that specifically recognizes a protein can be analyzed using in situ hybridization (e.g., sequential hybridization) and/or in situ sequencing (e.g., using padlock probes and rolling circle amplification of ligated padlock probes). Further, the reporter oligonucleotide of the labelling agent and/or a complement thereof and/or a product (e.g., a hybridization product, a ligation product, an extension product (e.g., by a DNA or RNA polymerase), a replication product, a transcription/reverse transcription product, and/or an amplification product) thereof can be recognized by another labelling agent and analyzed.

In some embodiments, an analyte (a nucleic acid analyte or non-nucleic acid analyte) can be specifically bound by two labelling agents (e.g., antibodies) each of which is attached to a reporter oligonucleotide (e.g., DNA) that can participate in ligation, replication, and sequence decoding reactions, e.g., using a probe or probe set (e.g. a padlock probe, a SNAIL probe set, a circular probe, or a padlock probe and a connector). In some embodiments, the probe set may comprise two or more probe oligonucleotides, each comprising a region that is complementary to each other. For example, a proximity ligation reaction can include reporter oligonucleotides attached to pairs of antibodies that can be joined by ligation if the antibodies have been brought in proximity to each other, e.g., by binding the same target protein (complex), and the DNA ligation products that form are then used to template PCR amplification, as described for example in Soderberg et al., Methods. (2008), 45 (3): 227-32, the entire contents of which are incorporated herein by reference. In some embodiments, a proximity ligation reaction can include reporter oligonucleotides attached to antibodies that each bind to one member of a binding pair or complex, for example, for analyzing a binding between members of the binding pair or complex. For detection of analytes using oligonucleotides in proximity, see, e.g., U.S. Patent Application Publication No. 2002/0051986, the entire contents of which are incorporated herein by reference. In some embodiments, two analytes in proximity can be specifically bound by two labelling agents (e.g., antibodies) each of which is attached to a reporter oligonucleotide (e.g., DNA) that can participate, when in proximity when bound to their respective targets, in ligation, replication, and/or sequence decoding reactions.

In some embodiments, one or more reporter oligonucleotides (and optionally one or more other nucleic acid molecules such as a connector) aid in the ligation of the probe. Upon ligation, the probe may form a circularized probe. In some embodiments, one or more suitable probes can be used and ligated, wherein the one or more probes comprise a sequence that is complementary to the one or more reporter oligonucleotides (or portion thereof). The probe may comprise one or more barcode sequences. In some embodiments, the one or more reporter oligonucleotide may serve as a primer for rolling circle amplification (RCA) of the circularized probe. In some embodiments, a nucleic acid other than the one or more reporter oligonucleotide is used as a primer for rolling circle amplification (RCA) of the circularized probe. For example, a nucleic acid capable of hybridizing to the circularized probe at a sequence other than sequence(s) hybridizing to the one or more reporter oligonucleotide can be used as the primer for RCA. In other examples, the primer in a SNAIL probe set is used as the primer for RCA.

In some embodiments, one or more analytes can be specifically bound by two primary antibodies, each of which is in turn recognized by a secondary antibody each attached to a reporter oligonucleotide (e.g., DNA). Each nucleic acid molecule can aid in the ligation of the probe to form a circularized probe. In some instances, the probe can comprise one or more barcode sequences. Further, the reporter oligonucleotide may serve as a primer for rolling circle amplification of the circularized probe. The nucleic acid molecules, circularized probes, and RCA products can be analyzed using any suitable method disclosed herein for in situ analysis.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some embodiments, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example ATP-dependent ligases, NAD+-dependent ligases, and RNA ligases. Some examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some embodiments, said ends are not ligated directly to each other, but instead occurs either via the intermediacy of one or more intervening (so-called “gap” or “gap-filling” (oligo) nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo) nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo) nucleotide, such that the gap (oligo) nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some embodiments, ligation stabilizes the hybridization complex containing the ligated polynucleotides prior to subsequent steps, comprising amplification and detection.

In some embodiments, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tm) of the DNA strands. This selectively reduces the concentration of annealed mismatched substrates (expected to have a slightly lower Tm around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

In some embodiments, the DNA probe comprises at least one gene-specific barcode. In some embodiments, the at least one barcode is specific for each of the target RNA molecules. In some embodiments, the target RNA molecules that are hybridized by the DNA probes are circularized by ligation.

### (v) Quantitation

Numerous techniques can be employed to assess the existence of a detectable marker, either on the detection probe itself or in combination with the analysis of cellular markers, to characterize the target cell under examination. To quantify the quantity of a detection probe or any other specific binding partner, a practical approach involves tagging it with a detectable moiety, such as a metal, fluorescent compound, luminescent substance, radioactive material, or enzymatically active entity.

Fluorescent moieties offer a versatile means of labeling nearly any biomolecule, structure, or cell type. Immunofluorescent moieties can be targeted to bind not only specific proteins but also specific conformations, cleavage products, or site modifications such as phosphorylation. Additionally, individual peptides and proteins can be genetically engineered to autofluoresce, exemplified by their expression as green fluorescent protein chimeras within cells (Jones et al., 1999, Trends Biotechnol. 17 (12): 477-81).

Mass cytometry is a modified form of flow cytometry that employs heavy metal ion tags instead of fluorochromes to label probes. The readout is achieved through time-of-flight mass spectrometry. This advancement enables the simultaneous combination of numerous specificities in a single sample without experiencing significant spillover between channels. For a relevant illustration, refer to the work of Bendall et al. (2011) published in Science 332 (6030): 687-696, which is explicitly incorporated by reference. An alternate approach for detecting metal labels is through scanning mass spectrometry, including but not limited to nano-SIMS.

By employing multiple fluorescent or metal labels on a single sample, it becomes feasible to quantitatively detect and perform simultaneous multiplex analysis. Numerous quantitative techniques have been devised to leverage the distinctive attributes of fluorescence. These techniques include direct fluorescence measurements, fluorescence resonance energy transfer (FRET), fluorescence polarization or anisotropy (FP), time-resolved fluorescence (TRF), fluorescence lifetime measurements (FLM), fluorescence correlation spectroscopy (FCS), and fluorescence photobleaching recovery (FPR).

Flow cytometry and mass cytometry serve as valuable tools for quantifying various parameters, such as the presence of cell surface proteins and their conformational or posttranslational modifications. They also allow the analysis of intracellular or secreted proteins when permeabilization facilitates antibody (or probe) access. Both single-cell multiparameter and multicell multiparameter multiplex assays are employed in the field. These assays utilize quantitative imaging, fluorescence, and confocal microscopy to identify input cell types and read the parameters. see Confocal Microscopy Methods and Protocols (Methods in Molecular Biology Vol. 122.) Paddock, Ed., Humana Press, 1998.

In some embodiments, the circularized target RNA molecules are amplified in situ by rolling circle amplification (RCA). In some embodiments, the amplified probes having the barcodes in DNA amplicons are detected. In some embodiments, at least one bridge probe is hybridized to at least one of the plurality of the amplified probes. In some embodiments, the at least one bridge probe is targeted by at least one fluorescent probe. In some embodiments, the at least one fluorescent probe is imaged by a plurality channel of a confocal microscope. In some embodiments, the bridge probes and the fluorescent probes are stripped away after imaging and re-hybridized to at least one of the plurality of DNA probes that are not previously hybridized. In some embodiments, the re-hybridized fluorescent probes are imaged by a plurality channel of a confocal microscope. In some embodiments, the imaging of the stripped and re-hybridized probes are repeated at least two times until all the barcodes are read. In some embodiments, each round of the imaging provides location information of each of the target RNA molecules hybridized with the DNA probes that are further hybridized with fluorescent probes.

In some embodiments, at least four target RNA molecules are identified from the amplified signals from said probes per one round of an imaging. In some embodiments, at least two rounds of the imaging are performed in the plant tissue to identify location of the target RNA molecules. In some embodiments, the spatially mapped genes collected from at least two rounds of imaging are mapped to locate the genes in the three dimensional gene expression map. In some embodiments, expression of at least 10 genes are spatially mapped after the at least two rounds of imaging in plant tissue. In some embodiments, expression of at least 100genes are spatially mapped after the at least two rounds of imaging in plant tissue. In some embodiments, expression of at least 1,000 genes are spatially mapped after the at least two rounds of imaging in plant tissue. In some embodiments, expression of at least 10,000 genes are spatially mapped after the at least two rounds of imaging in plant tissue.

In some embodiments, the method can spatially map at least one gene in plant tissue. In some embodiments, the plant tissue is whole-mount. In some embodiments, the plant tissue is sectioned. In some embodiments, the plant tissue is derived from rice, maize, soybean, or sorghum. In some embodiments, gene expression of a plurality of genes is spatially mapped in a plurality of plant cell types in the plant tissue. In some embodiments, the method allows the interrogation of spatial regulation of complex cellular responses in the plant tissue during its developmental stages and/or during its exposure to stress.

The present disclosure teaches that location of mRNA molecules is defined by the sequence-by-hybridization (SBH) chemistry that targets the barcode sequences of DNA amplicons across sequential rounds of probing, imaging, and stripping. In each imaging round, four targets are detected using each of the four channels of a confocal microscope. After imaging, fluorescent detection probes are stripped by incubating the sample in high concentration of formamide, and the next round of hybridization targets a new set of four genes. In order to avoid signal overcrowding in small cells of plant root tip, which will make it impossible to distinguish signal from different transcripts, the present disclosure teaches the targeting of four genes in each imaging round.

### (vi) Analyzing Spatial Data

The present disclosure provides image stacks from each round were registered in 3D based on the cell wall boundary staining information by a global affine alignment using random sample consensus (RANSAC)-based feature matching. The analysis pipeline of Bigstream (Wang et al., 2021, Cell) with modifications is adopted.

To analyze the spatial data at the single-cell level, cell segmentation has been performed on cell wall boundary stained images using PlantSeg, which performs deep learning-assisted cell boundary prediction and graph partitioning-based cell segmentation (Wolny et al., 2020, eLife).

The registered and segmented images has been used for downstream analysis with starfish, a Python library for processing image-based spatial transcriptomics data. Single molecule-derived spots are automatically detected and decoded based on their signal then assigned to cells and counted for each cell, resulting in cell-by-gene matrix, which can be analyzed in a similar way as single-cell RNA-seq data (but with 3D spatial information).

Kits

The present disclosure provides kits comprising one or more oligonucleotides disclosed herein, and reagents for performing the methods provided herein, for example reagents required for one or more steps comprising fixation, permeabilization, hybridization, ligation, amplification, and/or detection, as described herein. In some embodiments, the kit further comprises a target nucleic acid. In some embodiments, any or all of the polynucleotides are DNA molecules. In some embodiments, the target nucleic acid is a messenger RNA molecule.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some embodiments, the kit can also comprise any of the reagents described herein, e.g., wash buffer and ligation buffer. In some embodiments, the kits contain reagents for detection and/or imaging, such as barcode detection probes or detectable labels/markers/probes. In some embodiments, the kits optionally contain other components, for example nucleic acid primers, enzymes and reagents, buffers, nucleotides, modified nucleotides, reagents for additional assays.

In one aspect, the provided embodiments can be applied in an in situ method of analyzing nucleic acid sequences, such as an in situ transcriptomic analysis or in situ sequencing, for example from intact tissues or samples in which the spatial information has been preserved. In other aspects, the embodiments can be applied in an imaging or detection method for multiplexed, single-cell fluorescent gene expression analysis. In another aspect, the provided embodiments can be used to identify or detect regions of interest in target nucleic acids with their spatial information.

The present disclosure provides kits for spatially mapping at least one gene in plant tissue in situ. In some embodiments, the kit comprises (i) a fixative comprising a formalin, an acetic acid, and an alcohol, (ii) a mixture of cell wall penetrating enzymes, and/or (iii) a T4 DNA ligase. In some embodiments, the kit further comprises (iv) a plurality of DNA probes for a control gene, each of the DNA probes comprising a padlock probe and a primer, wherein the padlock probe and the primer have complementary sequences to form a circular structure, (v) a plurality of bridge probes, each of the bridge probes having a complementary sequence for the padlock probe, and/or (vi) a plurality of fluorescent probes, each of the fluorescent probes is hybridized with each of the bridge probes. In further embodiments, the kit encloses instructions on how to design the DNA probes, the bridge probes, and fluorescent probes, all of which are specific for a plurality of genes of interest.

### EXAMPLES

The present disclosure is further illustrated by the following examples that should not be construed as limiting. The contents of all references, patents, and published patent applications cited throughout this application, as well as the Figures, are incorporated herein by reference in their entirety for all purposes.

### Example 1: Methods for Multiplexed Single-Cell 3D Spatial Gene Expression Analysis in Plant Tissue

The current state of the art is to use single cell RNA-seq to discover where specific genes of interest or introduced transgenes are expressed. However, single cell clusters are devoid of any spatial context. For example, while it may be known that a gene or transgene is expressed, it is not known where in the tissue it is expressed which is important for many reasons, like validating whether an introduced transgene is recapitulating the normal pattern of expression of that gene. In contrast, the methods and procedures of the present disclosure provide a 3D context and does not require preparing many 2D sections to “see” in what cells the gene is expressed. Most plant researchers are not interested in a single gene but rather are interested in the suite of genes that for example may be induced by expression of a specific transgene.

### Sample Preparation

Arabidopsis thaliana accession Col-0 seeds (hereafter Arabidopsis) were sown on square plates containing Linsmaier and Skoog medium (Caisson Labs, catalogue no. LSP03) with 0.8% sucrose solidified with 1% agar (Caisson Labs, catalogue no. A038). Plates were kept vertically for 5 days in a growth chamber under an 8:16 h light/dark regime at 21° C.

Gene expression Map Experimental Procedure

Chemicals and enzymes The following chemicals and enzymes were used: a poly-D-lysine coated dish (MatTek, catalogue no. P35GC-1.5-14-C); T4 DNA ligase (Thermo Fisher Scientific, catalogue no. EL0011); EquiPhi29 DNA polymerase (Thermo Fisher Scientific, catalogue no. A39391); SUPERaseIn RNase inhibitor (Invitrogen, catalogue no. AM2696); aminoallyl dUTP (AnaSpec, catalogue no. AS-83203); Dulbecco's phosphate-buffered saline (DPBS) (Sigma, catalogue no. D8662); molecular biology grade BSA (New England Biolabs, catalogue no. B9000S); dNTPs (New England Biolabs, catalogue no. N0447S); Fluorescent Brightener 28 disodium salt solution (Sigma, catalogue no. 910090); formaldehyde solution for molecular biology, 36.5%-38% in water (Sigma, catalogue no. F8775); Triton-X (Sigma, catalogue no. 93443); Proteinase K (Invitrogen, catalogue no. 25530049); nuclease-free water (Invitrogen, catalogue no. AM9937); BS (PEG) 9 (Thermo Fisher Scientific, catalogue no. 21582); 20× SSC buffer (Sigma-Aldrich, catalogue no. S6639); ribonucleoside vanadyl complex (New England Biolabs, catalogue no. S1402S); formamide (Sigma, catalogue no. F9037); RNase-free Tris buffer pH 8.0 (Invitrogen, catalogue no. AM9855G); RNase-free EDTA pH 8.0 (Invitrogen, catalogue no. AM9260G); cellulase (Yaklut, catalogue no. YAKL0013); macerozyme (Yakult, catalogue no. YAKL0021); and pectinase (Thermo Fisher Scientific, catalogue no. ICN19897901).

Probe design Target genes were selected manually based on their cell type-specific expression. Probes were constructed by combining the probe design used in STARmap (Wang, X. et al. Science 361, eaat5691, 2018) and HYBISS (Gyllborg, D. et al. Nucleic Acids Res. 48, e112, 2020) (FIG. 3a). A SNAIL probe—a pair comprising a padlock probe (PLP) and a primer—was designed. (1) For each gene, 40-50-nucloetide sequences with a GC content of 40%-60% were selected and it was confirmed that there was no homologous region in the other transcripts by blasting against TAIR10 Arabidopsis genome. (2) Selected sequences were split into halves, each of 20-25 nucleotides (the 5′ halves for PLPs and the 3′ halves for primers), with a two-nucleotide gap between, ensuring that the melting temperature (Tm) of each half is around 60° C. (3) PLPs have complementary sequences for target specific bridge probes. (4) Four SNAIL probes were designed for each gene. (5) PLPs and primers have complementary sequences to form a circular structure. Bridge probes and detection read-out probes were designed as described in Gyllborg, D. et al. (Nucleic Acids Res. 48, e112, 2020). All probes were manufactured by Integrated DNA Technologies. SNAIL probes were manufactured in the form of oPools Oligo Pools with desalting purification. Bridge probes were manufactured individually with desalting purification. Detection read-out probes were manufactured individually with HPLC purification.

Sample fixation and permeabilization Five-day-old root tips were cut on the agar plate using a razor blade, mounted on a dry poly-D-lysine coated dish using tweezers, and immediately fixed, dehydrated and rehydrated in a manner similar to that described in Rozier et al. (Nat. Protoc. 9, 2464-2475, 2014) with modifications. The following steps were conducted on the dish. Arabidopsis root tips were immersed in FAA (16% v/v formaldehyde, 5% v/v acetic acid and 50% ethanol) for 1 h at room temperature. RNase-free water was used throughout the entire protocol. Samples were then dehydrated in a series of 10-min washes once in 70% (v/v in nuclease-free water) ethanol, once in 90% ethanol and twice in 100% ethanol, followed by two 10-min washes in 100% methanol, and then were stored in 100% methanol at −20° C. overnight. The next day, samples were rehydrated in a series of 5-min washes in 75% (v/v), 50% and 25% methanol in DPBS-T (0.1% Tween 20 in DPBS) at room temperature. The cell wall was partially digested by incubating samples in cell wall digestion solution (0.06% cellulase, 0.06% macerozyme, 0.1% pectinase, and 1% SUPERase in DPBS-T) for 5 min on ice, and then for 30 min at room temperature. After two washes in DPBS-TR (DPBS-T and 1% SUPERase), samples were fixed in 10% (v/v) formaldehyde for 30 min at room temperature and washed with DPBS-TR. Proteins were digested by incubating samples in protein digestion buffer (0.1 M Tris-HCl pH 8, 50 mM EDTA pH 8) with a 1:100 volume of Proteinase K (20 mg ml-1, RNA grade; Invitrogen, catalogue no. 25530049) for 30 min at 37° C. After two washes in DPBS-TR, samples were fixed in 10% (v/v) formaldehyde for 30 min at room temperature and washed with DPBS-TR.

SNAIL probe hybridization, amplification and fixation The following steps are based on STARmap protocols with modifications. A pool of SNAIL probes (500 nM each) was heated at 90°° C. for 5 min and cooled at room temperature. Samples were incubated in hybridization buffer (2× SSC, 30% formamide, 1% Triton-X, 20 mM ribonucleoside vanadyl complex and pooled SNAIL probes at 10 nM per oligo) in a 40° C. humidified oven overnight. After hybridization, samples were washed twice in DPBS-TR and once in 4× SSC in DPBS-TR for 30 min at 37° C. and rinsed with DPBS-TR at room temperature. Samples were then incubated in a T4 DNA ligation mixture (1:50 dilution of T4 DNA ligase supplemented with 1× BSA and 0.2 U μl-1 of SUPERase-In) at room temperature overnight. After ligation, samples were washed twice with DPBS-TR for 10 min at room temperature and incubated in a rolling circle amplification (RCA) mixture (1:20 dilution of equiPhi29 DNA polymerase, 250 μM dNTP, 0.1 μg μl-1 BSA, 1 mM dithiothreitol, 0.2 U μl-1 of SUPERase-In and 20 μM aminoallyl dUTP) at 37° C. overnight. After RCA, samples were rinsed in DPBS-T and covalently cross-linked with 4.3 μg μl-1 BS (PEG) 9 in DPBS-T. BS (PEG) 9 was then quenched by incubating samples in 1 M Tris-HCl (pH 8) for 30 min at room temperature.

Gel embedding and tissue clearing After the fixation of DNA amplicons, samples were embedded in acrylamide gel by incubating in a polymerization mixture (4% acrylamide, 0.2% bis-acrylamide, 0.1% ammonium persulfate and 0.1% tetramethylethylenediamine in DPBS-T) for 1.5 h at room temperature. Samples were then rinsed in DPBS-T. After gel embedding, samples were cleared by incubating in ClearSee (Kurihara, D. et al. Development 142, 4168-4179, 2015) at room temperature overnight.

Sequence-by-hybridization Samples were washed with 2× SSC for 5 min at room temperature and then incubated in a bridge probe hybridization mixture (2× SSC, 20% formamide and four bridge probes at 100 nM per oligo in water) for 1 h at room temperature. After washing twice in 2× SSC for 5 min at room temperature, samples were incubated in a detection probe hybridization mixture (2× SSC, 20% formamide, 1:100 dilution of Calcofluor White (Fluorescent Brightener 28 disodium salt solution) and fluorescent detection oligos at 100 nM per oligos in water) for 1 h at room temperature. Samples were washed in 2× SSC and ClearSee for 5 min at room temperature and stored in ClearSee until imaging. After imaging, the gene expression map signal was stripped by incubating in stripping buffer (65% formamide in 2× SSC) at 30° C. for 30 min.

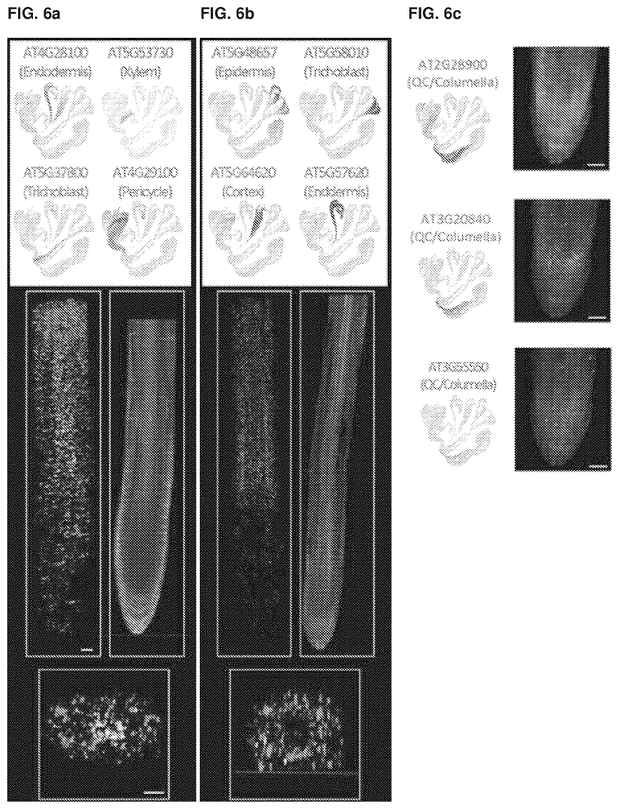

Imaging Imaging was performed using a Leica Stellaris 8 confocal microscope equipped with a DMi8 CS Premium, supercontinuum white light laser, laser 405 DMOD, power HyD detectors and an HC PL APO CS2×40/1.10 water objective. The image size for a field-of-view was 512×512 pixels with a voxel size of 0.57 μmp33 0.57 μm×0.42 μm, and three fields-of-view were acquired for each root sample unless otherwise stated. The 2D images shown in FIG. 6b were taken in a scan format of 2,048×2,048 pixels with denoising (averaging two images). The following channel settings were used: 405 nm excitation, 420-510 nm emission; 499 nm excitation, 504-554 nm emission; 554 nm excitation, 559-650 nm emission; 649 nm excitation, 657-735 nm emission; 752 nm excitation, 760-839 nm emission.

The gene expression map in the leaf Arabidopsis plants were grown in soil for 20 days with a 12 h light period. The fifth leaf (the largest) was used for the experiment. Leaves were processed as described above with slight modifications. Because the whole-mount leaf did not attach to the poly-D-lysine coated dish, the tissue was fixed in a 1.5 ml tube with FAA. A vacuum was applied to facilitate fixation. After the first fixation, the tissue was transferred to a poly-D-lysine coated dish and the downstream steps were carried out on the dish. The tissue was not embedded in the gel, because inventors did not perform multiple rounds of imaging. Before imaging, the tissue was mounted on a glass slide with a coverslip on top to immobilize the tissue. SNAIL probes for UBQ10 (AT4G05320) were used as described in Supplementary Table 2 of Nobori, T. et al., Nat. Plants 9, 1026-1033, 2023), which is incorporated by reference in its entirety.

### Gene Expression Map Data Processing

Image registration Sample handling could cause shifts in a field-of-view during image acquisition. To correct these shifts, image stacks from each round were registered in three dimensions based on the cell wall boundary staining information by a global affine alignment using random sample consensus-based feature matching (Fischler, M. A. et al. Commun. ACM 24, 381-395, 1981). Inventors adopted the analysis pipeline of Bigstream (Wang, Y. et al. Cell 184, 6361-6377, 2021) with modifications. The first round of images was used as a reference. The registered images were used for downstream analysis with starfish (github.com/spacetx/starfish), a Python library for processing image-based spatial transcriptomics data.

Spot detection and decoding Registered image stacks were processed with ImageJ (v.2.3.0) into individual images for each channel and z-step that starfish can process. Images were denoised using the Bandpass function, and the z axis was smoothed by Gaussian blurring using the GaussianLowPass function with the following parameters: lshort=0.5, llong=11 and threshold=0.0. Using the Clip function, an image clipping filter was applied to remove pixels of too low or too high intensity. Fluorescence in situ hybridization signals (spots) from single molecule-derived rolling circle products (RCP) were detected by a blob detection technique using the BlobDetector function, which is a multidimensional Gaussian spot detector that convolves kernels of multiple defined sizes with images to identify spots. The kernel sizes were determined based on the diameter of spots (typically around 1 μm). Detected spots were decoded based on the imaging round and the channel information using the SimpleLookupDecoder function.

Cell segmentation The cell wall staining image of the first imaging round (the same image used as a reference for image registration) was used for segmentation. PlantSeg (Wolny, A. et al. eLife 9, e57613, 2020) workflow was used to predict cell boundaries and label the cells in the image stacks. A re-scaling factor of [1.68, 2.28, 2.28] was used to fit our images to the ‘confocal PNAS_3d’ model on the software. A graphics processing unit-based convolutional neural network prediction was used for cell boundary prediction with the patch size of [80, 160, 160] and the ‘accurate’ mode (50% overlap between patches). The Multicut segmentation algorithm was used with under-/oversegmentation factor=0.5, 3D watershed, convolutional neural network predictions threshold=0.3, watershed seeds sigma=1.0, watershed boundary sigma=0, superpixels minimum size=1, and cell minimum size=1. After segmentation, images were re-scaled with the appropriate factors.

Spot assignment to segmented cells Based on the segmentation masks generated in the previous step, individual decoded spots were assigned to cells using the AssignTargets function. The spots were then counted for each target in each cell, resulting in a cell-by-gene matrix.

Image visualization Registered and decoded images were visualized using napari28, a fast, interactive, multidimensional image viewer for Python, by using the starfish function display.

### Gene Expression Map Count Data Analysis

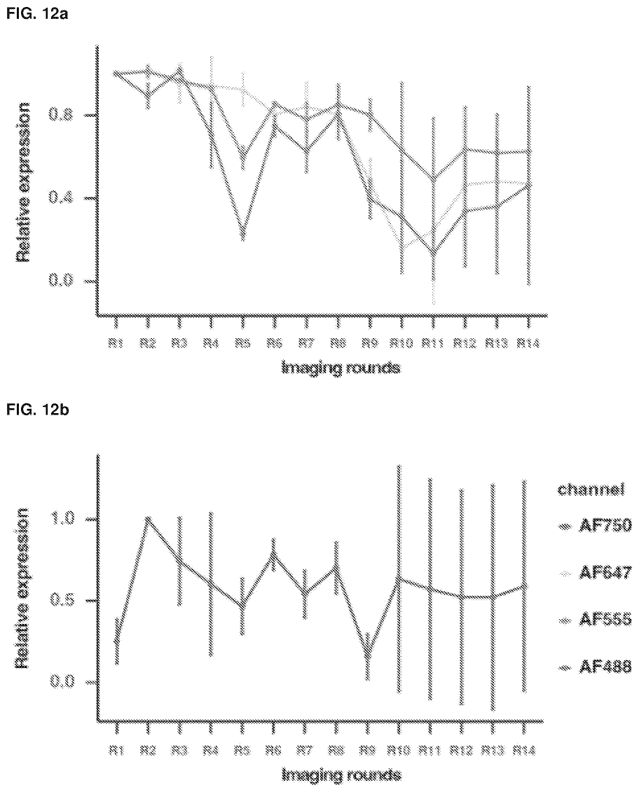

Scanpy was used for analyzing count data (Wolf, F. A. et al. Genome Biol. 19, 15, 2018). Cells that contain fewer than six spots (transcripts) were filtered out from the analysis. Count data were log-transformed, and principal components were calculated. A neighborhood graph was computed by using 10 principal components with a local neighborhood size of five. UMAP embedding was generated based on the neighborhood graph. Clustering was performed with the Leiden algorithm with a parameter resolution of 1. The plots in FIG. 12 were created using ggplot2 (v.3.3.5).

### HCR

HCR was performed as reported in Oliva, M. et al. (biorxiv.org/content/early/2022/03/04/2022.03.04.483008) with some modifications. Root tips were fixed and permeabilized as described above in the gene expression map method. After protein digestion and post fixation, the sample was pre-incubated in HCR probe hybridization buffer (Molecular Instruments, catalogue no. BPH02323) for 30 min at 37° C., then incurvated in HCR probe hybridization buffer with a 1:500 volume of a GFP-targeting probe mixture (designed by Molecular Instruments) overnight at 37° C. After probe hybridization, the sample was washed twice with HCR probe wash buffer (Molecular Instruments, catalogue BPH01923) for 30 min at 37° C. and twice with 5× SSCTR (5× SSC, 0.1% Tween and 0.2 U μl-1 of SUPERase-In) for 10 min at room temperature. The sample was then incubated in the HCR amplification buffer (Molecular Instruments, catalogue number BAM02323) for 30 min at room temperature. During the incubation, HCR amplifier B3-h1/2 Alexa Fluor 647 was heated to 95° C. for 90 s in a thermocycler and cooled at room temperature for 30 min. The amplification solution was prepared by adding a 1:50 volume of cooled HCR amplifiers to the HCR amplification buffer. The sample was incubated in the amplification solution overnight at room temperature and washed three times with 5× SSCTR for 20 min at room temperature. The sample was then cleared in ClearSee for more than 1 day until imaging. For imaging, the cell wall of the samples was stained with Calcofluor White as described above.

scRNA-seq Analysis

Processed and annotated data by Shahan et al. (Dev. Cell 57, 543-560, 2022). were downloaded from the Gene Expression Omnibus (GSE152766_Root_Atlas_spliced_unspliced_raw_counts.rds.gz). The R package Seurat (v.4.1.0) (Butler, et al. Nat. Biotech. 36, 411-420 (2018) was used to display the expression of target genes.

### Example 2. Accuracy of Gene Expression Map

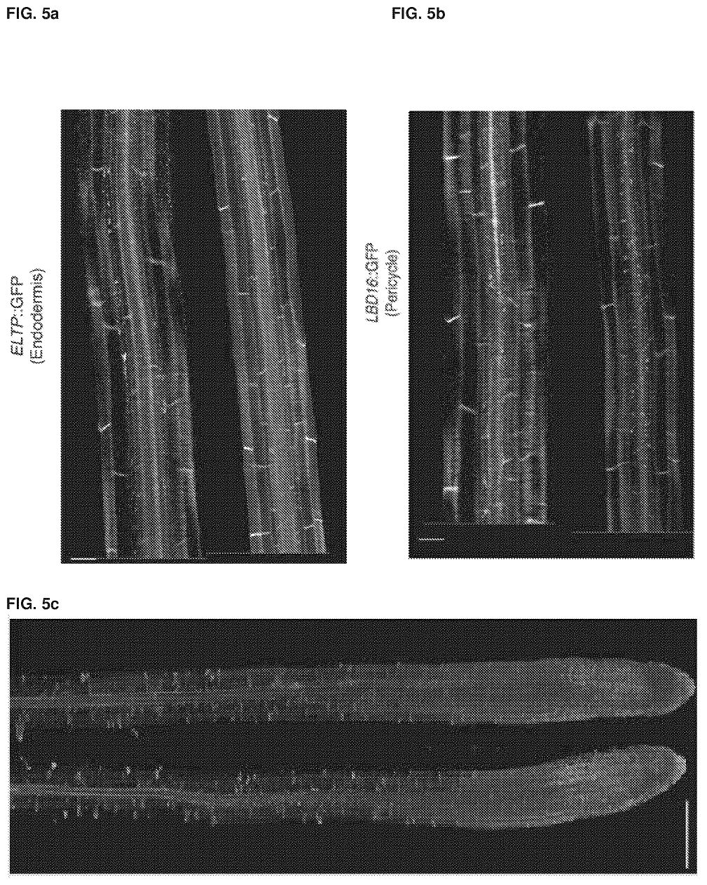

Inventors tested the accuracy of the gene expression map by comparing its signal with results from other imaging-based techniques. Inventors used transgenic Arabidopsis lines expressing green fluorescent protein (GFP) under the control of an endodermis-specific (EMBRYO LIPID TRANSFER PROTEIN or ELTP) or pericycle-specific (LATERAL ORGAN BOUNDARIES-DOMAIN 16 or LBD16) promoter. Cell type-specific GFP expression in these lines has been confirmed in a previous study Wyrsch, I. et al. New Phytol. 206, 774-784, 2015). We targeted the mRNA of GFP with the gene expression map and a hybridization chain reaction (HCR), which is also a hybridization-based approach recently applied to plant tissue. The gene expression map detected GFP mRNA in the expected cell types, which was further validated with HCR (FIGS. 5a and 5b). Together, these results confirmed the accuracy of the gene expression map.

### Example 3. Cell Type Marker Gene Mapping and Validation

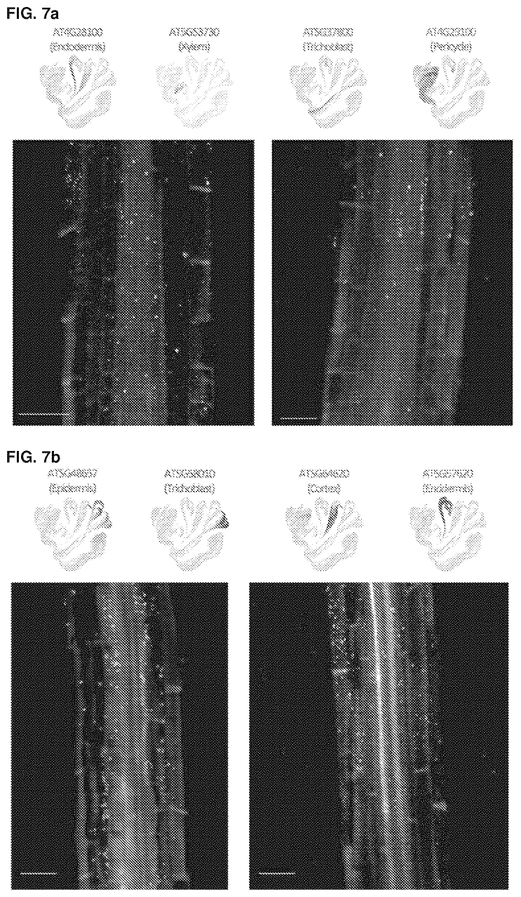

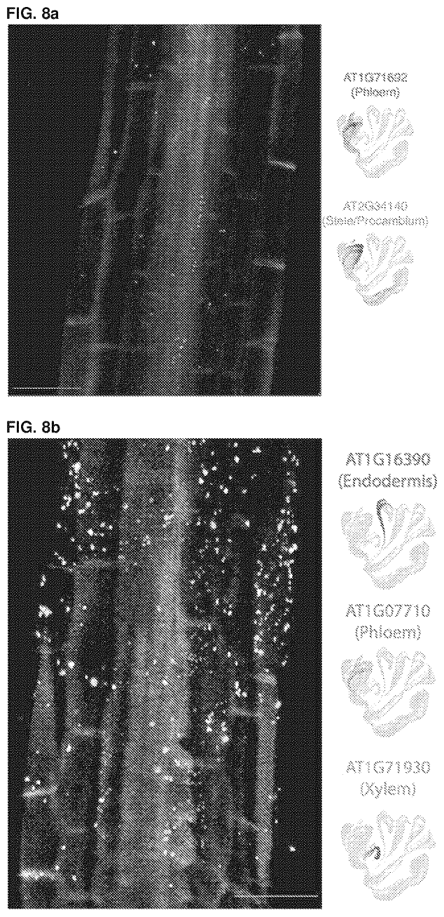

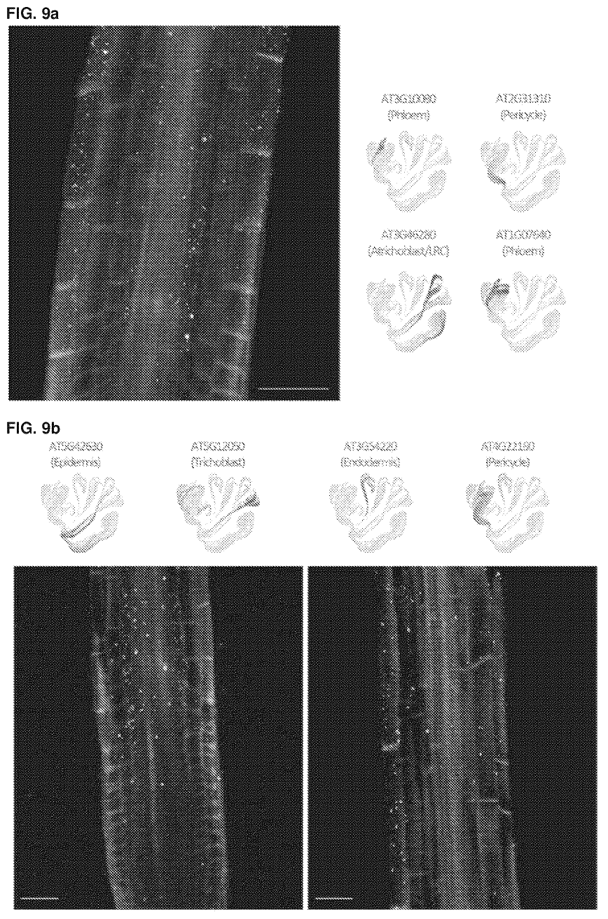

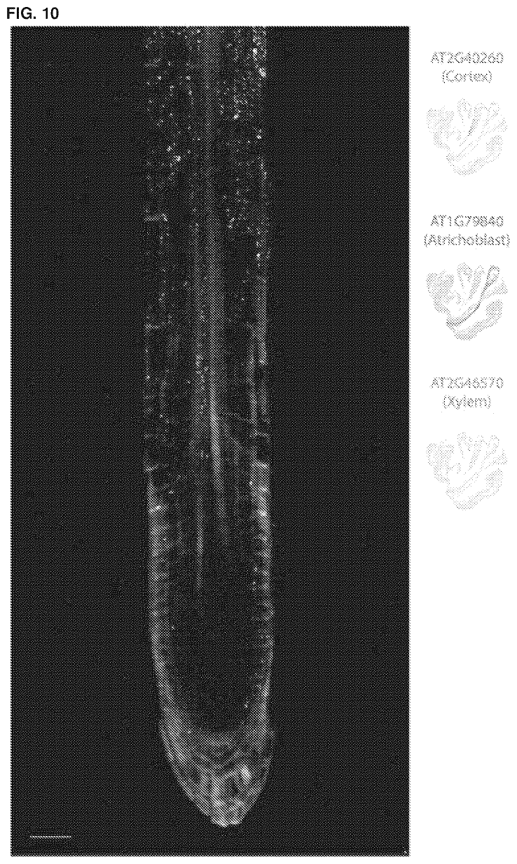

The gene expression map successfully mapped well-established/validated cell-type marker genes in expected cell types/regions in the root tip of Arabidopsis (FIG. 1d-1f and FIG. 6). The marker genes inventors targeted include AT4G28100 (ENDODERMIS7 or EN7; endodermis), AT4G29100 (BASIC HELIX LOOP HELIX 68 or BHLH68; pericycle), AT5G37800 (RHD SIX-LIKE 1 or RSL1; trichoblast), AT5G53730 (NDR1/HIN1-LIKE 26 or NHL26; xylem), AT5G57620 (MYB DOMAIN PROTEIN 36 or MYB36; endodermis), AT5G58010 (LJRHL1-LIKE 3 or LRL3; trichoblast) and AT3G54220 (SCARECROW or SCR; endodermis) (FIG. 1e-1f and FIG. 6; magnified images are provided in FIGS. 7 and 8). The gene expression map also validated cell type/region marker candidates predicted in a previous single-cell RNA-sequencing (scRNA-seq) study of Arabidopsis root tips (Shahan, R. et al. Dev. Cell 57, 543-560, 2022). For instance, AT3G46280 was detected in the root cap and elongating epidermis as predicted in the scRNA-seq data (FIG. 1e). Genes enriched in meristematic (AT5G42630) and elongation (AT5G12050) zones in the scRNA-seq data were mapped in the expected regions (FIG. 1f); AT5G12050 signal was detected in epidermis and vasculature, as shown in scRNA-seq (FIG. 1f). Quiescent center (QC) and columella signal was also detected from the marker genes AT2G28900, AT3G20840 and AT3G55550 (FIG. 6c). Other genes that are not shown in FIG. 1 are shown in FIGS. 9 and 10. Taken together, the gene expression map can be used as an efficient tool for validating marker genes identified in scRNA-seq data without generating transgenic plants.

### Example 4. Multiplexing Single-Cell Gene Expression

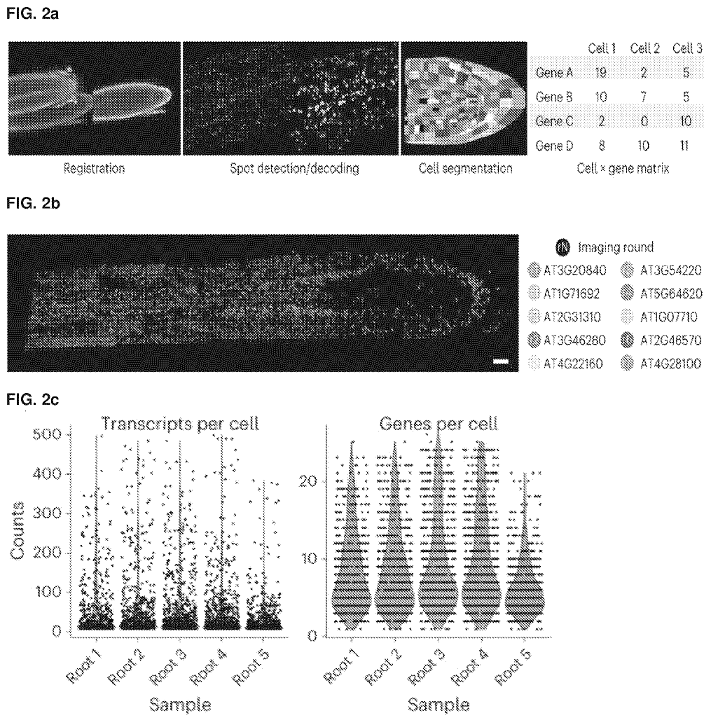

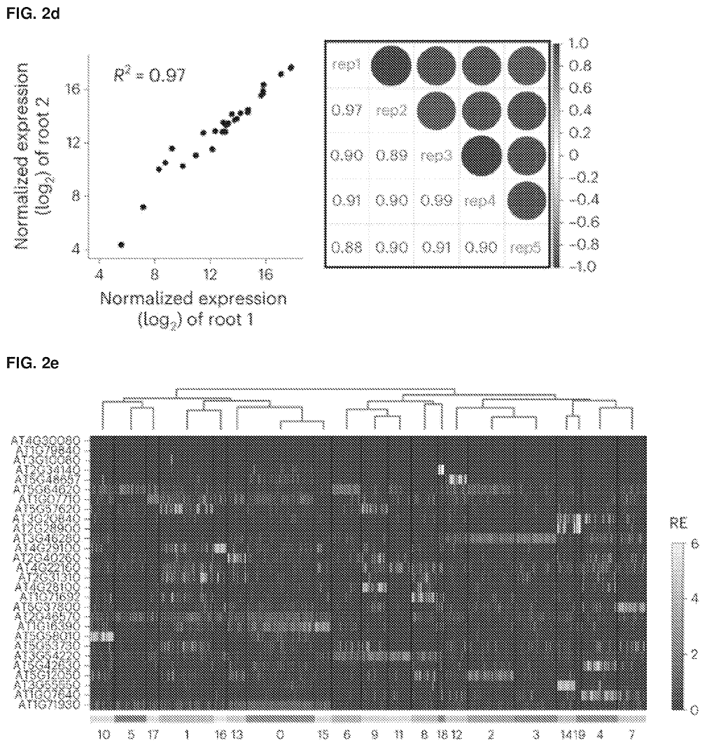

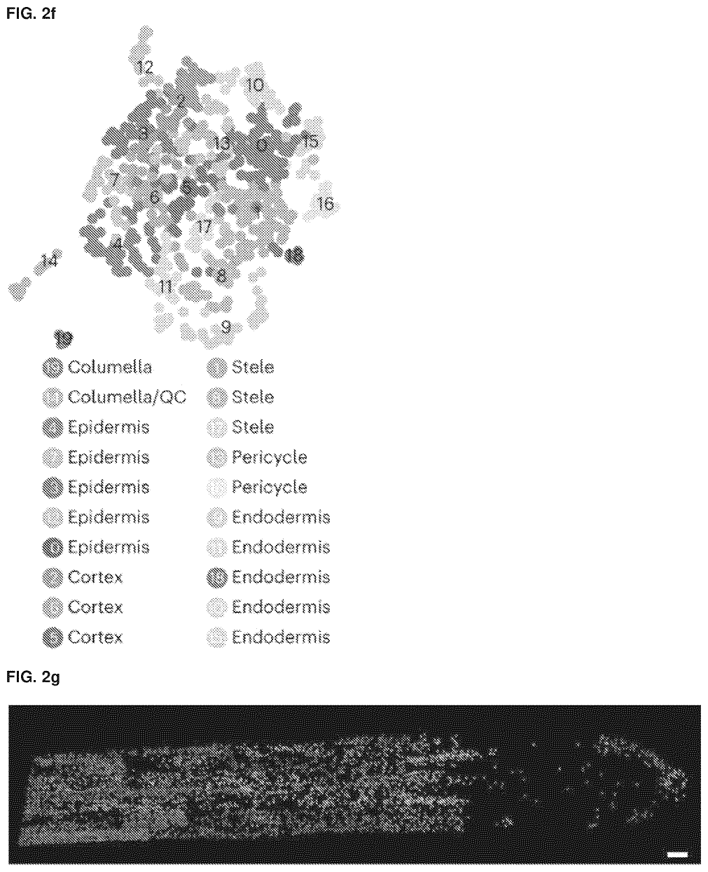

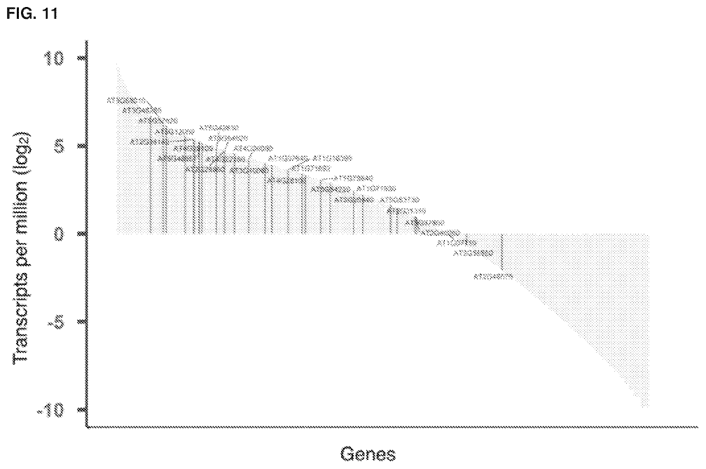

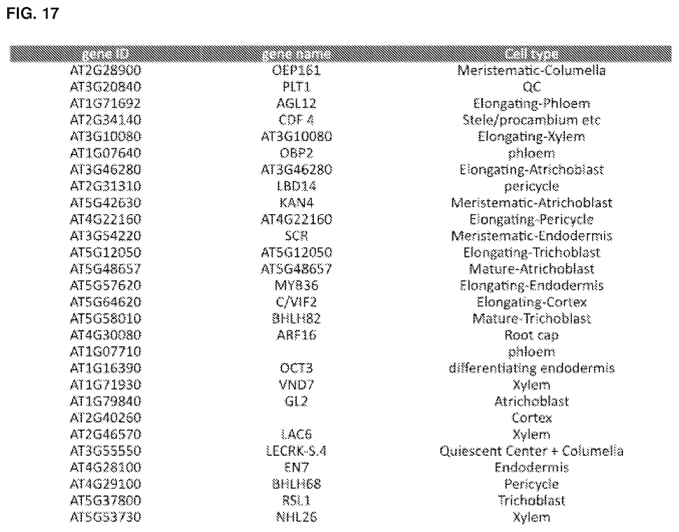

To demonstrate the multiplexing capacity of this method, inventors simultaneously targeted 28 genes in the same root tips with seven rounds of imaging. The targeted genes include known cell-type marker genes as well as unvalidated cell-type marker candidates identified in the scRNA-seq data (a full list is given in FIG. 17), which showed varying levels of expression in the root tip (FIG. 11). Inventors developed a computational pipeline to integrate whole-mount images from each imaging round and analyse gene expression at the single-cell resolution (FIG. 2a). Cell wall boundary information was obtained together with the RNA-derived signal in each imaging round to facilitate this process. The analysis pipeline first registers 3D images across imaging rounds using cell boundary information, automatically detects spots derived from single mRNA molecules and annotates spots with gene names. A merged image with detected and decoded transcripts successfully captured the cell-type architecture of the root tip (FIG. 2b). To analyse the spatial data at the single-cell level, cell segmentation was performed based on cell wall boundary information using PlantSeg, which performs deep learning-assisted cell boundary prediction and graph partitioning-based cell segmentation (Wolny, A. et al. eLife 9, e57613, 2020) (FIG. 2a). Annotated spots were assigned to individual cells and counted, resulting in a cell-by-gene matrix, a standard scRNA-seq data form that can be used for clustering and dimension reduction analyses (FIG. 2a).

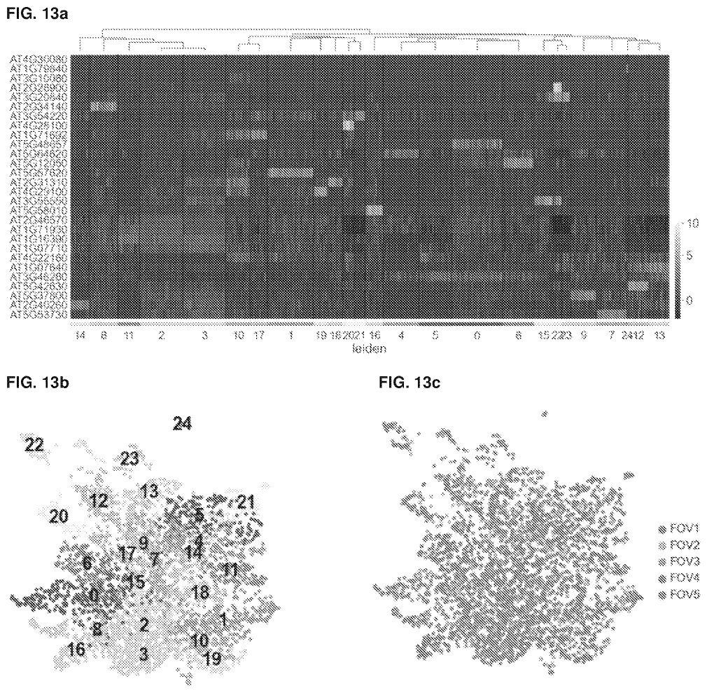

Inventors analyzed five root tip preparations and identified a total of 259,781 RNA molecules from 3,608 cells (median 19 molecules per cell) (FIG. 2c). The assays were highly robust and reproducible, detecting comparable numbers of transcripts for each RNA species between different biological samples (FIG. 2d). This suggests that gene expression between cells or samples can be compared quantitatively. Hierarchical clustering and heatmap visualization revealed cell population-specific expression of target genes (FIG. 2e and FIG. 13a). Genes that showed low expression in a previous RNA-sequencing study were detected successfully (FIG. 2e and FIG. 11), suggesting a high sensitivity of the gene expression map. Inventors performed de novo clustering using the gene expression map data and visualized the data on Uniform Manifold Approximation and Projection (UMAP) without using any spatial information (FIG. 2f and FIG. 13b-13c). These clusters successfully captured major cell types and developmental stages in the root tip (FIG. 2g). Together, these results demonstrate that the gene expression map can spatially map dozens of genes at a single-cell resolution in a highly reproducible manner.

### Example 5. Limitations of Gene Expression Map

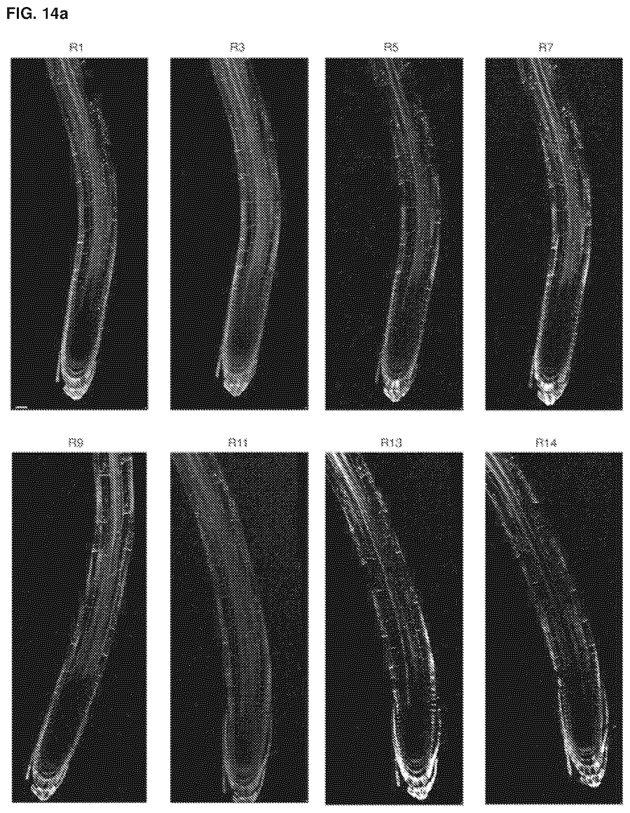

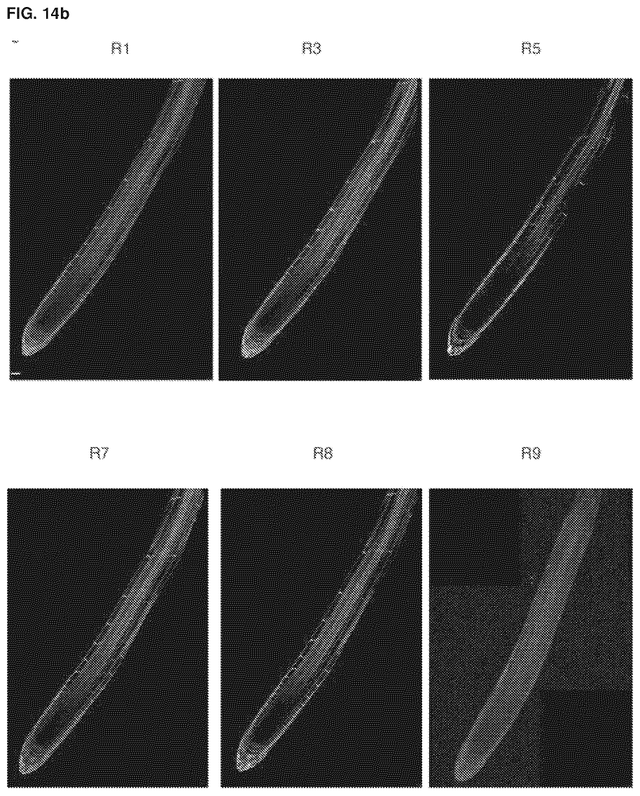

To test the limits of the gene expression map, inventors performed 14 successive rounds of experiments targeting the same genes. Inventors observed qualitatively consistent signals across the imaging rounds (FIG. 14), except for one detection fluorophore (Alexa Fluor 750), whose signal decayed after the eighth round, indicating that the current protocol can detect 50 genes in the same tissue. The results also indicate that the order of imaging rounds would not substantially affect the qualitative readouts, at least in the first eight rounds. Quantitative analysis of gene expression across 14 rounds showed an overall decreasing signal and increasing noise over imaging rounds (FIG. 12a-12b). Improving the accuracy and sensitivity of spot detection is an important future task.

### Example 6. Features of The Gene Expression Map

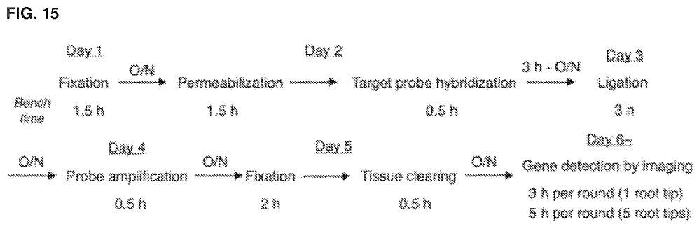

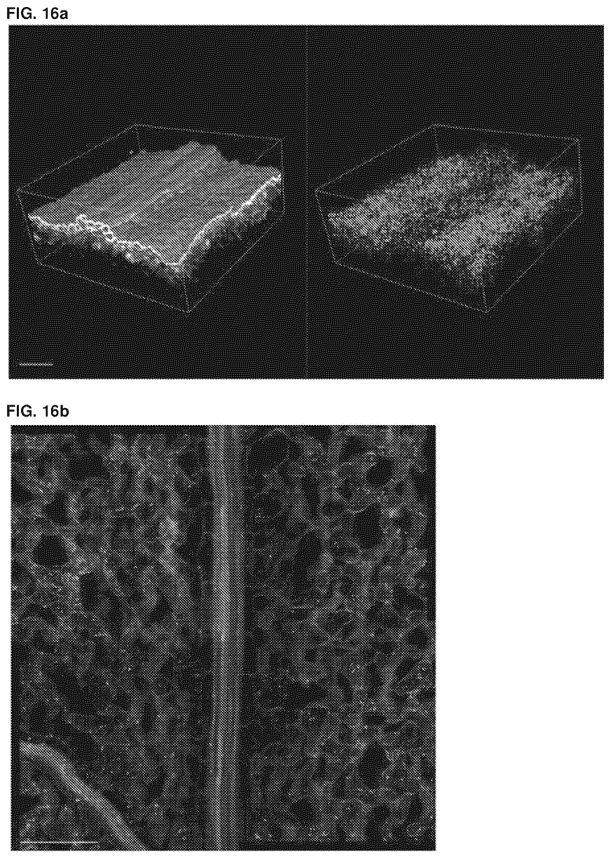

The gene expression map is a new technology that enables multiplexed single-cell spatial gene expression analysis in whole-mount plant tissue without requiring transgenic plant lines. A gene expression map experiment can be performed on a timescale similar to other in situ hybridization protocols in Arabidopsis; sample preparation takes 4-5 days with ˜10 h total bench time (FIG. 15a). Imaging can be performed using a regular confocal microscope. Each imaging round takes 3 h for one root tip and 5 h for five root tips in the current study; thus 21 h and 35 h to finish imaging for a 28-gene experiment in one and five root tips, respectively. It is possible to image much larger tissues with longer imaging times. Signal could be detected from the maturation zone of the root (FIG. 5c). The gene expression map also successfully detected a housekeeping gene (POLYUBIQUITIN 10 or UBQ10) in whole-mount Arabidopsis leaves (FIG. 16).

Inventors demonstrated that the current protocol can detect 50 genes in the same tissue. Previous studies have shown that more than 25 rounds of imaging are possible with DNA amplicons obtained using approaches similar to our method (Lee et al., 2015), suggesting that the gene expression map can potentially target more than 100 genes with optimized protocols.

A recent study successfully reconstructed 3D spatial expression of the transcriptome of Arabidopsis flower meristems by integrating scRNA-seq data with validated spatial expression of 28 genes using novoSpaRc (Neumann, M. et al. Nat. Commun. 13, 2838, 2022; Nitzan, M. et al., Nature 576, 132-137, 2019). The gene expression map, combined with such computational approaches, can generate a 3D spatial transcriptome atlas of various tissues and conditions. Discriminating highly similar transcripts is challenging with hybridization-based methods like the gene expression map, but the computational approach described above can compensate for this limitation. The transgene-free nature of the gene expression map makes this technology potentially applicable to any plant species. Cell-type annotation in scRNA-seq is challenging in many crop plants because their marker genes are often not conserved in other well-characterized species such as Arabidopsis. A potential challenge in applying the gene expression map to other plant species is permeabilization of the tissue, which can be achieved by optimizing cell wall degradation protocols (Giacomello, S. et al. Nat. Protoc. 13, 2425-2446, 2018). The gene expression map will become a widely used tool for efficient cluster annotation in scRNA-seq studies of a variety of plant species. Beyond cell typing, the gene expression map will offer unique opportunities to interrogate spatial regulation of complex cellular responses in plant tissue during stress and development with the ability to directly tap into various mutants that already exist.

### Further Numbered Embodiments of the Disclosure

Other subject matter contemplated by the present disclosure is set out in the following numbered embodiments:
1. A method of spatially mapping gene expression of a plurality of genes in plant tissue in situ, the method comprising:
   a. fixing a plant tissue with a fixative;
   b. permeabilizing the plant tissue;
   c. hybridizing a plurality of DNA probes with target RNA molecules transcribed from at least one gene;
   d. amplifying said probes having barcodes by rolling circle amplification (RCA);
   e. detecting a plurality of amplified signals from said probes by a sequence-by-hybridization (SBH) chemistry, thereby identifying location of the target RNA molecules; and
   f. obtaining three dimensional gene expression map with the plurality of the genes.
2. The method of embodiment 1, wherein the target RNA molecules are expressed in the identified location.
3. The method of embodiment 1, wherein the fixative comprises a formalin, an acetic acid, and an alcohol.
4. The method of embodiment 3, wherein the alcohol is an ethanol.
5. The method of embodiment 1, wherein a cell wall of the plant tissue is permeabilized with a cell wall degradation enzyme.
6. The method of embodiment 5, wherein the cell wall degradation enzyme is a cellulase, a macerozme, pectinase, a glycosyl-dyrolase, an oxidoreductase, a lyase, an esterase, a polygalaturonase, a protease, or a xylanase.
7. The method of embodiments 5-6, wherein the cell wall degradation enzyme is a cellulase, a macerozme, pectinase, or combinations thereof.
8. The method of embodiment 1, wherein the DNA probe comprises at least one gene-specific barcode.
9. The method of embodiment 1, wherein the at least one barcode is specific for each of the target RNA molecules.
10. The method of embodiment 1, wherein the target RNA molecules that are hybridized by the DNA probes are circularized by ligation.
11. The method of embodiment 10, wherein the circularized target RNA molecules are amplified in situ by rolling circle amplification (RCA).
12. The method of embodiment 1, wherein the amplified probes having the barcodes in DNA amplicons are detected.
13. The method of embodiment 12, wherein at least one bridge probe is hybridized to at least one of the plurality of the amplified probes.
14. The method of embodiment 13, wherein the at least one bridge probe is targeted by at least one fluorescent probe.
15. The method of embodiment 14, wherein the at least one fluorescent probe is imaged by a plurality channel of a confocal microscope.
16. The method of embodiment 15, wherein the bridge probes and the fluorescent probes are stripped away after imaging and re-hybridized to at least one of the plurality of DNA probes that are not previously hybridized.
17. The method of embodiment 16, wherein the re-hybridized fluorescent probes are imaged by a plurality channel of a confocal microscope.
18. The method of embodiment 17, wherein the imaging of the stripped and re-hybridized probes are repeated at least two times until all the barcodes are read.
19. The method of embodiment 18, wherein each round of the imaging provides location information of each of the target RNA molecules hybridized with the DNA probes that are further hybridized with fluorescent probes.
20. The method of embodiment 19, wherein each round of the imaging detects expression of at least two genes.
21. The method of embodiment 1, wherein at least four target RNA molecules are identified from the amplified signals from said probes per one round of an imaging.
22. The method of embodiment 21, wherein at least two rounds of the imaging are performed in the plant tissue to identify location of the target RNA molecules.
23. The method of embodiment 22, wherein the spatially mapped genes collected from at least two rounds of imaging are mapped to locate the genes in the three dimensional gene expression map.
24. The method of embodiment 23, wherein expression of at least 10 genes are spatially mapped after the at least two rounds of imaging in plant tissue.
25. The method of embodiment 23, wherein expression of at least 100 genes are spatially mapped after the at least two rounds of imaging in plant tissue.
26. The method of embodiment 23, wherein expression of at least 1,000 genes are spatially mapped after the at least two rounds of imaging in plant tissue.
27. The method of embodiment 23, wherein expression of at least 10,000 genes are spatially mapped after the at least two rounds of imaging in plant tissue.
28. The method of embodiment 1, wherein the plant tissue is whole-mount or sectioned.
29. The method of embodiment 1, wherein the gene expression of a plurality of genes is spatially mapped in a plurality of plant cell types in the plant tissue.
30. The method of embodiment 1, wherein the method allows the interrogation of spatial regulation of complex cellular responses in the plant tissue during its developmental stages and/or during its exposure to stress.
31. A kit for spatially mapping gene expression of a plurality of genes in plant tissue in situ, the kit comprising:
   a. a fixative comprising a formalin, an acetic acid, and an alcohol;
   b. a mixture of cell wall penetrating enzymes;
   c. a T4 DNA ligase;
   d. a plurality of DNA probes for a control gene, each of the DNA probes comprising a padlock probe and a primer, wherein the padlock probe and the primer have complementary sequences to form a circular structure;
   e. a plurality of bridge probes, each of the bridge probes having a complementary sequence for the padlock probe; and
   f. a plurality of fluorescent probes, each of the fluorescent probes is hybridized with each of the bridge probes.
32. The kit of embodiment 31, further comprising instructions on how to design the DNA probes, the bridge probes, and fluorescent probes, all of which are specific for a plurality of genes of interest.
33. The kit of embodiment 31, wherein the alcohol is an ethanol.
34. The kit of embodiment 31, wherein the cell wall degradation enzyme is a cellulase, a macerozme, pectinase, a glycosyl-dyrolase, an oxidoreductase, a lyase, an esterase, a polygalaturonase, a protease, or a xylanase.
35. The kit of embodiment 31 or 34, wherein the cell wall degradation enzyme is a cellulase, a macerozme, pectinase, or combinations thereof.
36. The kit of embodiment 31, wherein the padlock probe comprises at least one gene-specific barcode.
37. The kit of embodiment 31, wherein the at least one barcode is specific for each of target RNA molecules transcribed from the at least one gene.
38. The kit of embodiment 37, wherein the target RNA molecules that are hybridized by the DNA probes are circularized by the T4 DNA ligase.
39. The kit of embodiment 38, wherein the circularized target RNA molecules are amplified in situ by rolling circle amplification (RCA).
40. The kit of embodiment 39, wherein the amplified probes having barcodes in DNA amplicons are detected by SBH chemistry.
41. The kit of embodiment 31, wherein at least one bridge probe is hybridized to at least one of the plurality of the amplified probes.
42. The kit of embodiment 41, wherein the at least one bridge probe is targeted by at least one fluorescent probe.
43. The kit of embodiment 31, wherein at least one fluorescent probe is imaged by a plurality channel of a confocal microscope.
44. The kit of embodiment 43, wherein the bridge probes and the fluorescent probes are stripped away after imaging and re-hybridized to at least one of the plurality of DNA probes that are not previously hybridized.
45. The kit of embodiment 31, wherein the re-hybridized fluorescent probes are imaged by a plurality channel of a confocal microscope.
46. The kit of embodiment 43, wherein the imaging of the stripped and re-hybridized probes are repeated at least two times until all the barcodes are read.
47. The kit of embodiment 46, wherein each round of the imaging provides location information of each of the target RNA molecules hybridized with the DNA probes that are further hybridized with fluorescent probes.
48. The kit of embodiment 46, wherein each round of the imaging detects expression of at least two genes.
49. The kit of embodiment 1, wherein at least four target RNA molecules are identified from the amplified signals from said probes per one round of an imaging.
50. The kit of embodiment 49, wherein at least two rounds of the imaging are performed in the plant tissue to identify location of the target RNA molecules.
51. The kit of embodiment 50, the spatially mapped genes collected from at least two rounds of imaging are mapped to locate the genes in the three dimensional gene expression map.
52. The kit of embodiment 51, wherein expression of at least 10 genes are spatially mapped after the at least two rounds of imaging in plant tissue.
53. The kit of embodiment 51, wherein expression of at least 100 genes are spatially mapped after the at least two rounds of imaging in plant tissue.
54. The kit of embodiment 51, wherein expression of at least 1,000 genes are spatially mapped after the at least two rounds of imaging in plant tissue.
55. The kit of embodiment 51, wherein expression of at least 10,000 genes are spatially mapped after the at least two rounds of imaging in plant tissue.
56. The kit of embodiment 31, wherein the plant tissue is whole-mount or sectioned.
57. The kit of embodiment 31, wherein the kit enables to detect gene expression of a plurality of genes that is spatially mapped in a plurality of plant cell types in the plant tissue.
58. A method of analyzing multiplexed, three-dimensional spatial gene expression in plant tissue, the method comprising:
   a. fixing a plant tissue with a fixative;
   b. permeabilizing the plant tissue;
   c. hybridizing a plurality of DNA probes with target RNA molecules transcribed from at least one gene;
   d. amplifying said probes having barcodes by rolling circle amplification (RCA);
   e. detecting a plurality of amplified signals from said probes by a sequence-by-hybridization (SBH) chemistry, thereby identifying location of the target RNA molecules; and
   f. obtaining three dimensional gene expression map with the plurality of the genes.
59. The method of embodiment 58, wherein the target RNA molecules are expressed in the identified location.
60. The method of embodiment 58, wherein the fixative comprises a formalin, an acetic acid, and an alcohol.
61. The method of embodiment 60, wherein the alcohol is an ethanol.
62. The method of embodiment 58, wherein a cell wall of the plant tissue is permeabilized with a cell wall degradation enzyme.
63. The method of embodiment 62, wherein the cell wall degradation enzyme is a cellulase, a macerozme, pectinase, a glycosyl-dyrolase, an oxidoreductase, a lyase, an esterase, a polygalaturonase, a protease, or a xylanase.
64. The method of embodiments 62-63, wherein the cell wall degradation enzyme is a cellulase, a macerozme, pectinase, or combinations thereof.
65. The method of embodiment 58, wherein the DNA probe comprises at least one gene-specific barcode.
66. The method of embodiment 58, wherein the at least one barcode is specific for each of the target RNA molecules.
67. The method of embodiment 58, wherein the target RNA molecules that are hybridized by the DNA probes are circularized by ligation.
68. The method of embodiment 67, wherein the circularized target RNA molecules are amplified in situ by rolling circle amplification (RCA).
69. The method of embodiment 58, wherein the amplified probes having the barcodes in DNA amplicons are detected.
70. The method of embodiment 69, wherein at least one bridge probe is hybridized to at least one of the plurality of the amplified probes.
71. The method of embodiment 70, wherein the at least one bridge probe is targeted by at least one fluorescent probe.
72. The method of embodiment 71, wherein the at least one fluorescent probe is imaged by a plurality channel of a confocal microscope.
73. The method of embodiment 72, wherein the bridge probes and the fluorescent probes are stripped away after imaging and re-hybridized to at least one of the plurality of DNA probes that are not previously hybridized.
74. The method of embodiment 73, wherein the re-hybridized fluorescent probes are imaged by a plurality channel of a confocal microscope.
75. The method of embodiment 74, wherein the imaging of the stripped and re-hybridized probes are repeated at least two times until all the barcodes are read.
76. The method of embodiment 75, wherein each round of the imaging provides location information of each of the target RNA molecules hybridized with the DNA probes that are further hybridized with fluorescent probes.
77. The method of embodiment 76, wherein each round of the imaging detects expression of at least two genes.
78. The method of embodiment 58, wherein at least four target RNA molecules are identified from the amplified signals from said probes per one round of an imaging.
79. The method of embodiment 78, wherein at least two rounds of the imaging are performed in the plant tissue to identify location of the target RNA molecules.
80. The method of embodiment 79, the spatially mapped genes collected from at least two rounds of imaging are mapped to locate the genes in the three dimensional gene expression map.
81. The method of embodiment 80, wherein expression of at least 10 genes are spatially mapped after the at least two rounds of imaging in plant tissue.
82. The method of embodiment 80, wherein expression of at least 100 genes are spatially mapped after the at least two rounds of imaging in plant tissue.
83. The method of embodiment 80, wherein expression of at least 1,000 genes are spatially mapped after the at least two rounds of imaging in plant tissue.
84. The method of embodiment 80, wherein expression of at least 10,000 genes are spatially mapped after the at least two rounds of imaging in plant tissue.
85. The method of embodiment 58, wherein the plant tissue is whole-mount or sectioned.
86. The method of embodiment 58, wherein gene expression of a plurality of genes is spatially mapped in a plurality of plant cell types in the plant tissue.
87. The method of embodiment 58, wherein the method allows the interrogation of spatial regulation of complex cellular responses in the plant tissue during its developmental stages and/or during its exposure to stress.

### INCORPORATION BY REFERENCE

All references, articles, publications, patents, patent publications, and patent applications cited herein within the above text and/or cited below are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

### REFERENCES

1. Cole, B. et al. Plant Single-Cell Solutions for Energy and the Environment.
2. Seyfferth, C. et al. Advances and Opportunities of Single-Cell Transcriptomics for Plant Research. Annu. Rev. Plant Biol. 1-20 (2021).
3. Birnbaum, K. D. Power in numbers: Single-cell RNA-seq strategies to dissect complex tissues. Annu. Rev. Genet. 52, 203-221 (2018).
4. Rao, A., Barkley, D., França, G. S. & Yanai, I. Exploring tissue architecture using spatial transcriptomics. Nature 596, 211-220 (2021).
5. Giacomello, S. et al. Spatially resolved transcriptome profiling in model plant species. Nat Plants 3, 17061 (2017).
6. Laureyns, R. et al. An in situ sequencing approach maps PLASTOCHRONI at the boundary between indeterminate and determinate cells. Plant Physiol. 188, 782-794 (2022).
7. Xia, K. et al. The single-cell stereo-seq reveals region-specific cell subtypes and transcriptome profiling in Arabidopsis leaves. Dev. Cell (2022) doi: 10.1016/j.devcel.2022.04.011.
8. Hejátko, J. et al. In situ hybridization technique for mRNA detection in whole mount Arabidopsis samples. Nat. Protoc. 1, 1939-1946 (2006).
9. Brewer, P. B., Heisler, M. G., Hejátko, J., Friml, J. & Benková, E. In situ hybridization for mRNA detection in Arabidopsis tissue sections. Nat. Protoc. 1, 1462-1467 (2006).
10. Moses, L. & Pachter, L. Museum of spatial transcriptomics. Nat. Methods 19, 534-546 (2022).
11. Gyllborg, D. et al. Hybridization-based in situ sequencing (HybISS) for spatially resolved transcriptomics in human and mouse brain tissue. Nucleic Acids Res. 48, e112-e112 (2020).
12. Wendrich, J. R. et al. Vascular transcription factors guide plant epidermal responses to limiting phosphate conditions. Science 370, 777-782 (2020).
13. Menand, B. et al. An ancient mechanism controls the development of cells with a rooting function in land plants. Science 316, 1477-1480 (2007).
14. Shahan, R. et al. A single-cell Arabidopsis root atlas reveals developmental trajectories in wild-type and cell identity mutants. Dev. Cell (2022) doi: 10.1016/j.devcel.2022.01.008.
15. Wolny, A. et al. Accurate and versatile 3D segmentation of plant tissues at cellular resolution. Elife 9, e57613 (2020).
16. Lee, J. H. et al. Fluorescent in situ sequencing (FISSEQ) of RNA for gene expression profiling in intact cells and tissues. Nat. Protoc. (2015) doi: 10.1038/nprot.2014.191.
17. Wang, X. et al. Three-dimensional intact-tissue sequencing of single-cell transcriptional states. Science 361, (2018).
18. Rozier, F., Mirabet, V., Vernoux, T. & Das, P. Analysis of 3D gene expression patterns in plants using whole-mount RNA in situ hybridization. Nat. Protoc. 9, 2464-2475 (2014).
19. Oliva, M. et al. An environmentally-responsive transcriptional state modulates cell identities during root development. bioRxiv 2022.03.04.483008 (2022) doi: 10.1101/2022.03.04.483008.
20. Kurihara, D., Mizuta, Y., Sato, Y. & Higashiyama, T. ClearSee: A rapid optical clearing reagent for whole-plant fluorescence imaging. Development 142, 4168-4179 (2015).
21. Fischler, M. A. & Bolles, R. C. Random sample consensus. Commun. ACM 24, 381-395 (1981).
22. Wang, Y. et al. EASI-FISH for thick tissue defines lateral hypothalamus spatio-molecular organization. Cell (2021) doi: 10.1016/j.cell.2021.11.024.
23. Wolf, F. A., Angerer, P. & Theis, F. J. SCANPY: Large-scale single-cell gene expression data analysis. Genome Biol. 19, 1-5 (2018).
24. Lee J H, Daugharthy E R, Scheiman J, Kalhor R, Ferrante T C, Terry R, Turczyk B M, Yang J L, Lee H S, Aach J, Zhang K, Church G M. Fluorescent in situ sequencing (FISSEQ) of RNA for gene expression profiling in intact cells and tissues. Nat Protoc. 2015 March; 10 (3): 442-58.
25. Nobori, T., Oliva, M., Lister, R. et al. Multiplexed single-cell 3D spatial gene expression analysis in plant tissue using PHYTOMap. Nat. Plants 9, 1026-1033 (2023).

1. A method of spatially mapping gene expression of a plurality of genes in plant tissue in situ, the method comprising:
a. fixing a plant tissue with a fixative;
b. permeabilizing the plant tissue;
c. hybridizing a plurality of DNA probes comprising at least one barcode with target RNA molecules transcribed from at least one gene;
d. amplifying the probes by rolling circle amplification (RCA);
e. detecting a plurality of amplified signals from the probes by a sequence-by-hybridization (SBH) chemistry, thereby identifying location of the target RNA molecules; and
f. obtaining a three-dimensional gene expression map of a plurality of genes.

2. The method of claim 1, wherein the target RNA molecules are expressed in the identified location.

3. The method of claim 1, wherein the fixative comprises a formalin, an acetic acid, and an alcohol.

4. The method of claim 3, wherein the alcohol is an ethanol.

5. The method of claim 1, wherein a cell wall of the plant tissue is permeabilized with a cell wall degradation enzyme.

6. The method of claim 5, wherein the cell wall degradation enzyme is a cellulase, a macerozyme, a pectinase, a glycosyl-dyrolase, an oxidoreductase, a lyase, an esterase, a polygalaturonase, a protease, of a xylanase, or any combination of a cellulase, a macerozyme, and a pectinase.

7.-8. (canceled)

9. The method of claim 1, wherein the at least one barcode is specific for each of the target RNA molecules.

10. The method of claim 1, wherein the target RNA molecules that are hybridized by the DNA probes are circularized by ligation.

11. The method of claim 10, wherein the circularized target RNA molecules are amplified in situ by rolling circle amplification (RCA).

12. (canceled)

13. The method of claim 121, wherein at least one bridge probe is hybridized to at least one of the plurality of the amplified probes.

14. The method of claim 13, wherein the at least one bridge probe is targeted by at least one fluorescent probe.

15. The method of claim 14, wherein the at least one fluorescent probe is imaged by a plurality channel of a confocal microscope.

16. The method of claim 15, wherein the bridge probes and the fluorescent probes are stripped away after imaging and re-hybridized to at least one of the plurality of the amplified probes that are not previously hybridized.

17. The method of claim 16, wherein the re-hybridized fluorescent probes are imaged by a plurality channel of a confocal microscope.

18. The method of claim 17, wherein the imaging of the stripped and re-hybridized probes are repeated at least two times until all the barcodes are read.

19. The method of claim 18, wherein each round of the imaging provides location information of each of the target RNA molecules hybridized with the DNA probes that are further hybridized with fluorescent probes.

20. The method of claim 19, wherein each round of the imaging detects expression of at least two genes.

21. The method of claim 1, wherein at least four target RNA molecules are identified from the amplified signals from said probes per one round of an imaging.

22. The method of claim 21, wherein at least two rounds of the imaging are performed in the plant tissue to identify location of the target RNA molecules.

23. The method of claim 22, wherein the spatially mapped genes collected from at least two at least two-rounds of imaging are mapped to locate the genes in the three dimensional gene expression map.

24. The method of claim 23, wherein expression of at least 10 genes are spatially mapped after the at least two rounds of imaging in plant tissue,
wherein expression of at least 100 genes are spatially mapped after the at least two rounds of imaging in plant tissue,
wherein expression of at least 1,000 genes are spatially mapped after the at least two rounds of imaging in plant tissue, or
wherein expression of at least 10,000 genes are spatially mapped after the at least two rounds of imaging in plant tissue.

25.-27. (canceled)

28. The method of claim 1, wherein the plant tissue is whole-mount or sectioned.

29. The method of claim 1, wherein a plurality of gene expression is spatially mapped in a plurality of plant cell types in whole-mount plant tissue.

30. The method of claim 1, wherein the method allows the interrogation of spatial regulation of complex cellular responses in the plant tissue during its developmental stages and/or during its exposure to stress.

31. A kit for spatially mapping gene expression of a plurality of genes in plant tissue in situ, the kit comprising:
a. a fixative comprising a formalin, an acetic acid, and an alcohol;
b. a mixture of cell wall penetrating enzymes;
c. a T4 DNA ligase;
d. a plurality of DNA probes for a control gene, each of the DNA probes comprising a padlock probe and a primer, wherein the padlock probe and the primer have complementary sequences to form a circular structure;
e. a plurality of bridge probes, each of the bridge probes having a complementary sequence for the padlock probe; and
f. a plurality of fluorescent probes, each of the fluorescent probes is hybridized with each of the bridge probes.

32.-57. (canceled)


SEQUENCE LISTING (1 sequence)

SEQ ID NO: 1 - DNA, 12 bp, synthetic construct
  source 1..12: mol_type = other DNA
taatgttatctt